SEQUENCE LISTING (169 sequences)

SEQ ID NO: 1 - protein, 7 aa, Artificial Sequence
  Synthetic peptide
SRPEGAR

SEQ ID NO: 2 - protein, 7 aa, Artificial Sequence
  Synthetic peptide
NATYGPG

SEQ ID NO: 3 - protein, 7 aa, Artificial Sequence
  Synthetic peptide
SSSGGHK

SEQ ID NO: 4 - protein, 7 aa, Artificial Sequence
  Synthetic peptide
GGRLTPQ

SEQ ID NO: 5 - protein, 7 aa, Artificial Sequence
  Synthetic peptide
TPGRLVM

SEQ ID NO: 6 - protein, 7 aa, Artificial Sequence
  Synthetic peptide
TRVVEGK

SEQ ID NO: 7 - protein, 7 aa, Artificial Sequence
  Synthetic peptide
GTVRPVV

SEQ ID NO: 8 - protein, 7 aa, Artificial Sequence
  Synthetic peptide
GRPATTG

SEQ ID NO: 9 - protein, 7 aa, Artificial Sequence
  Synthetic peptide
ARSGPSS

SEQ ID NO: 10 - protein, 7 aa, Artificial Sequence
  Synthetic peptide
FKVDSTK

SEQ ID NO: 11 - protein, 7 aa, Artificial Sequence
  Synthetic peptide
DNKTILR

SEQ ID NO: 12 - protein, 7 aa, Artificial Sequence
  Synthetic peptide
SAPGSRL

SEQ ID NO: 13 - protein, 7 aa, Artificial Sequence
  Synthetic peptide
KREERML

SEQ ID NO: 14 - protein, 7 aa, Artificial Sequence
  Synthetic peptide
DVTRTEG

SEQ ID NO: 15 - protein, 7 aa, Artificial Sequence
  Synthetic peptide
TPGSHGR

SEQ ID NO: 16 - protein, 7 aa, Artificial Sequence
  Synthetic peptide
SGRGQPS

SEQ ID NO: 17 - protein, 7 aa, Artificial Sequence
  Synthetic peptide
GGGARTG

SEQ ID NO: 18 - protein, 7 aa, Artificial Sequence
  Synthetic peptide
NSVRSAG

SEQ ID NO: 19 - protein, 7 aa, Artificial Sequence
  Synthetic peptide
QGGRPSL

SEQ ID NO: 20 - protein, 7 aa, Artificial Sequence
  Synthetic peptide
APRKGTA

SEQ ID NO: 21 - protein, 7 aa, Artificial Sequence
  Synthetic peptide
SEGRRQT

SEQ ID NO: 22 - protein, 7 aa, Artificial Sequence
  Synthetic peptide
VPVGPRK

SEQ ID NO: 23 - protein, 7 aa, Artificial Sequence
  Synthetic peptide
SNGAGRG

SEQ ID NO: 24 - protein, 7 aa, Artificial Sequence
  Synthetic peptide
VLAGGVR

SEQ ID NO: 25 - protein, 7 aa, Artificial Sequence
  Synthetic peptide
DSVRSLV

SEQ ID NO: 26 - protein, 7 aa, Artificial Sequence
  Synthetic peptide
RTSERGP

SEQ ID NO: 27 - protein, 7 aa, Artificial Sequence
  Synthetic peptide
GQVGGSR

SEQ ID NO: 28 - protein, 7 aa, Artificial Sequence
  Synthetic peptide
GNGPSRL

SEQ ID NO: 29 - protein, 7 aa, Artificial Sequence
  Synthetic peptide
GAQSKSK

SEQ ID NO: 30 - protein, 7 aa, Artificial Sequence
  Synthetic peptide
GPGARGG

SEQ ID NO: 31 - protein, 7 aa, Artificial Sequence
  Synthetic peptide
SVVDMTR

SEQ ID NO: 32 - protein, 7 aa, Artificial Sequence
  Synthetic peptide
GRAAGSA

SEQ ID NO: 33 - protein, 7 aa, Artificial Sequence
  Synthetic peptide
GRTGREA

SEQ ID NO: 34 - protein, 7 aa, Artificial Sequence
  Synthetic peptide
ANAAALR

SEQ ID NO: 35 - protein, 7 aa, Artificial Sequence
  Synthetic peptide
KGGPAGS

SEQ ID NO: 36 - protein, 7 aa, Artificial Sequence
  Synthetic peptide
RRSAEVA

SEQ ID NO: 37 - protein, 7 aa, Artificial Sequence
  Synthetic peptide
RGQTVVG

SEQ ID NO: 38 - protein, 7 aa, Artificial Sequence
  Synthetic peptide
SAPNKLK

SEQ ID NO: 39 - protein, 7 aa, Artificial Sequence
  Synthetic peptide
LGGSAAR

SEQ ID NO: 40 - protein, 7 aa, Artificial Sequence
  Synthetic peptide
RSGRNAE

SEQ ID NO: 41 - protein, 7 aa, Artificial Sequence
  Synthetic peptide
TQASTLR

SEQ ID NO: 42 - protein, 7 aa, Artificial Sequence
  Synthetic peptide
GEPSRVR

SEQ ID NO: 43 - protein, 7 aa, Artificial Sequence
  Synthetic peptide
AGVRPAL

SEQ ID NO: 44 - protein, 7 aa, Artificial Sequence
  Synthetic peptide
SSERSRQ

SEQ ID NO: 45 - protein, 7 aa, Artificial Sequence
  Synthetic peptide
SGGERER

SEQ ID NO: 46 - protein, 7 aa, Artificial Sequence
  Synthetic peptide
KSSSSSG

SEQ ID NO: 47 - protein, 7 aa, Artificial Sequence
  Synthetic peptide
GGSRPGQ

SEQ ID NO: 48 - protein, 7 aa, Artificial Sequence
  Synthetic peptide
VGRGSAP

SEQ ID NO: 49 - protein, 7 aa, Artificial Sequence
  Synthetic peptide
ARGSGTP

SEQ ID NO: 50 - protein, 7 aa, Artificial Sequence
  Synthetic peptide
RGTTQGS

SEQ ID NO: 51 - protein, 7 aa, Artificial Sequence
  Synthetic peptide
SKGGPGG

SEQ ID NO: 52 - protein, 7 aa, Artificial Sequence
  Synthetic peptide
SKAGVGV

SEQ ID NO: 53 - protein, 7 aa, Artificial Sequence
  Synthetic peptide
VAGRAAG

SEQ ID NO: 54 - protein, 7 aa, Artificial Sequence
  Synthetic peptide
SRVGGGS

SEQ ID NO: 55 - protein, 7 aa, Artificial Sequence
  Synthetic peptide
GADGERR

SEQ ID NO: 56 - protein, 7 aa, Artificial Sequence
  Synthetic peptide
TAANPKS

SEQ ID NO: 57 - protein, 7 aa, Artificial Sequence
  Synthetic peptide
RADDVPR

SEQ ID NO: 58 - protein, 7 aa, Artificial Sequence
  Synthetic peptide
RSASSQA

SEQ ID NO: 59 - protein, 7 aa, Artificial Sequence
  Synthetic peptide
RGSSGQV

SEQ ID NO: 60 - protein, 7 aa, Artificial Sequence
  Synthetic peptide
VRGSSSP

SEQ ID NO: 61 - protein, 7 aa, Artificial Sequence
  Synthetic peptide
LRGGAQG

SEQ ID NO: 62 - protein, 7 aa, Artificial Sequence
  Synthetic peptide
MKSSGSA

SEQ ID NO: 63 - protein, 7 aa, Artificial Sequence
  Synthetic peptide
EASGGGR

SEQ ID NO: 64 - protein, 7 aa, Artificial Sequence
  Synthetic peptide
VQTGKSS

SEQ ID NO: 65 - protein, 7 aa, Artificial Sequence
  Synthetic peptide
GEGSGGR

SEQ ID NO: 66 - protein, 7 aa, Artificial Sequence
  Synthetic peptide
GGKAGGA

SEQ ID NO: 67 - protein, 7 aa, Artificial Sequence
  Synthetic peptide
ARGAGAA

SEQ ID NO: 68 - protein, 7 aa, Artificial Sequence
  Synthetic peptide
VGSQKSA

SEQ ID NO: 69 - protein, 7 aa, Artificial Sequence
  Synthetic peptide
AKGSGAP

SEQ ID NO: 70 - protein, 7 aa, Artificial Sequence
  Synthetic peptide
DDRTTAR

SEQ ID NO: 71 - protein, 7 aa, Artificial Sequence
  Synthetic peptide
ESGGRAP

SEQ ID NO: 72 - protein, 7 aa, Artificial Sequence
  Synthetic peptide
KADVKVP

SEQ ID NO: 73 - protein, 7 aa, Artificial Sequence
  Synthetic peptide
PGKSGVG

SEQ ID NO: 74 - protein, 7 aa, Artificial Sequence
  Synthetic peptide
RGAAAGS

SEQ ID NO: 75 - protein, 7 aa, Artificial Sequence
  Synthetic peptide
GADAHVR

SEQ ID NO: 76 - protein, 7 aa, Artificial Sequence
  Synthetic peptide
GGGKQAG

SEQ ID NO: 77 - protein, 7 aa, Artificial Sequence
  Synthetic peptide
GEGGGGR

SEQ ID NO: 78 - protein, 7 aa, Artificial Sequence
  Synthetic peptide
SKTGSTV

SEQ ID NO: 79 - protein, 7 aa, Artificial Sequence
  Synthetic peptide
SDGGGGR

SEQ ID NO: 80 - protein, 7 aa, Artificial Sequence
  Synthetic peptide
DGGGAAR

SEQ ID NO: 81 - protein, 7 aa, Artificial Sequence
  Synthetic peptide
ARTGTQG

SEQ ID NO: 82 - protein, 7 aa, Artificial Sequence
  Synthetic peptide
KGTGGSG

SEQ ID NO: 83 - protein, 7 aa, Artificial Sequence
  Synthetic peptide
SSSKTGS

SEQ ID NO: 84 - protein, 7 aa, Artificial Sequence
  Synthetic peptide
KAGGSQG

SEQ ID NO: 85 - protein, 7 aa, Artificial Sequence
  Synthetic peptide
RSGGPSL

SEQ ID NO: 86 - protein, 7 aa, Artificial Sequence
  Synthetic peptide
GGLAKSG

SEQ ID NO: 87 - protein, 7 aa, Artificial Sequence
  Synthetic peptide
SVEGGGR

SEQ ID NO: 88 - protein, 7 aa, Artificial Sequence
  Synthetic peptide
TRGPGGA

SEQ ID NO: 89 - protein, 7 aa, Artificial Sequence
  Synthetic peptide
EGVGRER

SEQ ID NO: 90 - protein, 7 aa, Artificial Sequence
  Synthetic peptide
RSGGVPG

SEQ ID NO: 91 - protein, 7 aa, Artificial Sequence
  Synthetic peptide
TTKGSGA

SEQ ID NO: 92 - protein, 7 aa, Artificial Sequence
  Synthetic peptide
KGERQAV

SEQ ID NO: 93 - protein, 7 aa, Artificial Sequence
  Synthetic peptide
SRGTASS

SEQ ID NO: 94 - protein, 7 aa, Artificial Sequence
  Synthetic peptide
RANPSPT

SEQ ID NO: 95 - protein, 7 aa, Artificial Sequence
  Synthetic peptide
GSGTKVS

SEQ ID NO: 96 - protein, 7 aa, Artificial Sequence
  Synthetic peptide
DDPRAAR

SEQ ID NO: 97 - protein, 7 aa, Artificial Sequence
  Synthetic peptide
KASGAGG

SEQ ID NO: 98 - protein, 7 aa, Artificial Sequence
  Synthetic peptide
GTTGPVK

SEQ ID NO: 99 - protein, 7 aa, Artificial Sequence
  Synthetic peptide
SGEGVTR

SEQ ID NO: 100 - protein, 7 aa, Artificial Sequence
  Synthetic peptide
KGSGGGI

SEQ ID NO: 101 - protein, 7 aa, Artificial Sequence
  Synthetic peptide
SDVTRVW

SEQ ID NO: 102 - protein, 7 aa, Artificial Sequence
  Synthetic peptide
YDSTRAG

SEQ ID NO: 103 - protein, 7 aa, Artificial Sequence
  Synthetic peptide
GQHRSYH

SEQ ID NO: 104 - protein, 7 aa, Artificial Sequence
  Synthetic peptide
QGKPGPW

SEQ ID NO: 105 - protein, 7 aa, Artificial Sequence
  Synthetic peptide
KGGGFHG

SEQ ID NO: 106 - protein, 7 aa, Artificial Sequence
  Synthetic peptide
SVASTRA

SEQ ID NO: 107 - protein, 7 aa, Artificial Sequence
  Synthetic peptide
EARPRAW

SEQ ID NO: 108 - protein, 7 aa, Artificial Sequence
  Synthetic peptide
IDVTRNM

SEQ ID NO: 109 - protein, 7 aa, Artificial Sequence
  Synthetic peptide
LLATTRG

SEQ ID NO: 110 - protein, 7 aa, Artificial Sequence
  Synthetic peptide
SKQAGGG

SEQ ID NO: 111 - protein, 7 aa, Artificial Sequence
  Synthetic peptide
LRHGMVA

SEQ ID NO: 112 - protein, 7 aa, Artificial Sequence
  Synthetic peptide
GRAAGGS

SEQ ID NO: 113 - protein, 7 aa, Artificial Sequence
  Synthetic peptide
RQDTHPR

SEQ ID NO: 114 - protein, 7 aa, Artificial Sequence
  Synthetic peptide
GGRPGSW

SEQ ID NO: 115 - protein, 7 aa, Artificial Sequence
  Synthetic peptide
DRSLRAL

SEQ ID NO: 116 - protein, 7 aa, Artificial Sequence
  Synthetic peptide
CMQGRAC

SEQ ID NO: 117 - protein, 7 aa, Artificial Sequence
  Synthetic peptide
RAGLEAV

SEQ ID NO: 118 - protein, 7 aa, Artificial Sequence
  Synthetic peptide
RGDGLGR

SEQ ID NO: 119 - protein, 7 aa, Artificial Sequence
  Synthetic peptide
RDRTREL

SEQ ID NO: 120 - protein, 7 aa, Artificial Sequence
  Synthetic peptide
RRDETRT

SEQ ID NO: 121 - protein, 7 aa, Artificial Sequence
  Synthetic peptide
ADMTRRG

SEQ ID NO: 122 - protein, 7 aa, Artificial Sequence
  Synthetic peptide
MYGVKMG

SEQ ID NO: 123 - protein, 7 aa, Artificial Sequence
  Synthetic peptide
RGDSWRG

SEQ ID NO: 124 - protein, 7 aa, Artificial Sequence
  Synthetic peptide
RGDGRWS

SEQ ID NO: 125 - protein, 7 aa, Artificial Sequence
  Synthetic peptide
SLGSTKV

SEQ ID NO: 126 - protein, 7 aa, Artificial Sequence
  Synthetic peptide
QDVTRHT

SEQ ID NO: 127 - protein, 7 aa, Artificial Sequence
  Synthetic peptide
RAKPGME

SEQ ID NO: 128 - protein, 7 aa, Artificial Sequence
  Synthetic peptide
LLGTTKA

SEQ ID NO: 129 - protein, 7 aa, Artificial Sequence
  Synthetic peptide
GDATRWG

SEQ ID NO: 130 - protein, 7 aa, Artificial Sequence
  Synthetic peptide
PGDGTRR

SEQ ID NO: 131 - protein, 7 aa, Artificial Sequence
  Synthetic peptide
EGRPGRW

SEQ ID NO: 132 - protein, 7 aa, Artificial Sequence
  Synthetic peptide
GRDYTRL

SEQ ID NO: 133 - protein, 7 aa, Artificial Sequence
  Synthetic peptide
LNGRGGQ

SEQ ID NO: 134 - protein, 7 aa, Artificial Sequence
  Synthetic peptide
ARGDGWR

SEQ ID NO: 135 - protein, 7 aa, Artificial Sequence
  Synthetic peptide
GRDVTRG

SEQ ID NO: 136 - protein, 7 aa, Artificial Sequence
  Synthetic peptide
SPSLRVL

SEQ ID NO: 137 - protein, 7 aa, Artificial Sequence
  Synthetic peptide
GRDVTRS

SEQ ID NO: 138 - protein, 7 aa, Artificial Sequence
  Synthetic peptide
TDRTRLA

SEQ ID NO: 139 - protein, 7 aa, Artificial Sequence
  Synthetic peptide
RGDGWSH

SEQ ID NO: 140 - protein, 7 aa, Artificial Sequence
  Synthetic peptide
RGGGVYG

SEQ ID NO: 141 - protein, 7 aa, Artificial Sequence
  Synthetic peptide
EVASTRG

SEQ ID NO: 142 - protein, 7 aa, Artificial Sequence
  Synthetic peptide
RLSEARI

SEQ ID NO: 143 - protein, 7 aa, Artificial Sequence
  Synthetic peptide
NMSRSGM

SEQ ID NO: 144 - protein, 7 aa, Artificial Sequence
  Synthetic peptide
RGDYPRS

SEQ ID NO: 145 - protein, 7 aa, Artificial Sequence
  Synthetic peptide
RRLADMG

SEQ ID NO: 146 - protein, 7 aa, Artificial Sequence
  Synthetic peptide
GSGQAGR

SEQ ID NO: 147 - protein, 7 aa, Artificial Sequence
  Synthetic peptide
VLAVKRM

SEQ ID NO: 148 - protein, 7 aa, Artificial Sequence
  Synthetic peptide
SDATRRW

SEQ ID NO: 149 - protein, 7 aa, Artificial Sequence
  Synthetic peptide
NPLGKVR

SEQ ID NO: 150 - protein, 7 aa, Artificial Sequence
  Synthetic peptide
IDVGSAD

SEQ ID NO: 151 - protein, 7 aa, Artificial Sequence
  Synthetic peptide
FAAMGSL

SEQ ID NO: 152 - protein, 7 aa, Artificial Sequence
  Synthetic peptide
PPYAVMV

SEQ ID NO: 153 - protein, 7 aa, Artificial Sequence
  Synthetic peptide
SRGSGPS

SEQ ID NO: 154 - protein, 7 aa, Artificial Sequence
  Synthetic peptide
AAKVAAP

SEQ ID NO: 155 - protein, 7 aa, Artificial Sequence
  Synthetic peptide
RSGVGSA

SEQ ID NO: 156 - DNA, 21 bp, Artificial Sequence
  Synthetic peptide
ctgggggggtcggcggcgcgt

SEQ ID NO: 157 - DNA, 21 bp, Artificial Sequence
  Synthetic peptide
aaggcgggggggtcgcagggg

SEQ ID NO: 158 - DNA, 21 bp, Artificial Sequence
  Synthetic peptide
attgatgttggtagtgcggat

SEQ ID NO: 159 - DNA, 21 bp, Artificial Sequence
  Synthetic peptide
ccgccgtatgcggtgatggtg

SEQ ID NO: 160 - DNA, 21 bp, Artificial Sequence
  Synthetic peptide
gcggcgaaggtggcggctccg

SEQ ID NO: 161 - DNA, 21 bp, Artificial Sequence
  Synthetic peptide
cggtctggtgttggtagtgct

SEQ ID NO: 162 - DNA, 21 bp, Artificial Sequence
  Synthetic peptide
tttgcggctatgggttcgttg

SEQ ID NO: 163 - DNA, 21 bp, Artificial Sequence
  Synthetic peptide
agtcggggttcggggccttct

SEQ ID NO: 164 - protein, 736 aa, adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 165 - protein, 735 aa, adeno-associated virus 2 [sequence omitted]

SEQ ID NO: 166 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 167 - protein, 748 aa, Artificial Sequence
  Synthetic polypeptide
  misc_feature (594)..(600): Xaa can be any naturally occurring amino acid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDSSAXXXXXXXASPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 168 - protein, 747 aa, Artificial Sequence
  Synthetic polypeptide
  misc_feature (591)..(597): Xaa can be any naturally occurring amino acid
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNAAAXXXXXXXAARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 169 - protein, 748 aa, Artificial Sequence
  Synthetic polypeptide
  misc_feature (592)..(598): Xaa can be any naturally occurring amino acid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAAAXXXXXXXASAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

(19) United States
(12) Patent Application Publication
(10) Pub. No.: US 2024/0197920 A1
(43) Pub. Date: Jun. 20, 2024

(54) ADENO-ASSOCIATED VIRAL VECTORS FOR TRANSDUCTION OF COCHLEA

(71) Applicant: THE CHILDREN'S HOSPITAL OF PHILADELPHIA, Philadelphia, PA (US)

(72) Inventors: Beverly DAVIDSON, Philadelphia, PA (US); Paul RANUM, Philadelphia, PA (US)

(73) Assignee: THE CHILDREN'S HOSPITAL OF PHILADELPHIA, Philadelphia, PA (US)

(21) Appl. No.: 18/557,033

(22) PCT Filed: Apr. 27, 2022
(86) PCT No.: PCT/US2022/026518

§ 371 Date: Oct. 24, 2023

Related U.S. Application Data
(60) Provisional application No. 63/180,394, filed on Apr. 27, 2021.

(51) Int. Cl. A61K 48/00; C12N 15/86

(52) U.S. Cl. CPC A61K 48/0058; C12N 15/86; C12N 2750/14122; C12N 2750/14143; C12N 2750/14145; C12N 2830/008

(57) ABSTRACT

Provided herein are compositions and methods for delivering a molecular therapeutic to the cochlea of a subject. The methods comprise administering an adeno-associated virus (AAV) to the cerebrospinal fluid of the subject. The AAVs encode a therapeutic transgene for molecular therapy. Optionally, the therapeutic transgene may be operably linked to a cochlea-specific promoter.

### REFERENCE TO RELATED APPLICATIONS

The present application claims the priority benefit of U.S. provisional application No. 63/180,394, filed Apr. 27, 2021, the entire contents of which are incorporated herein by reference.

### REFERENCE TO A SEQUENCE LISTING

The instant application contains a Sequence Listing, which has been submitted in ASCII format via EFS-Web and is hereby incorporated by reference in its entirety. Said ASCII copy, created on Apr. 20, 2022, is named CHOPP0048WO_ST25.txt and is 67,842 bytes in size.

### BACKGROUND

### 1. Field

The present disclosure relates generally to the fields of medicine and virology. More particularly, it concerns compositions and methods for delivery of molecular therapeutics to the cochlea.

### 2. Description of Related Art

The cochlear tissues of the inner ear have long been regarded as one of the most challenging tissues to access for delivery of molecular therapeutics. The cochlea is positioned inside the temporal bone, the densest bone in the human body. It occupies a series of winding passageways that house both the membranous labyrinth of the cochlea and the vestibular system. These membranous tissues are surrounded by bone, with the exception of three small openings: the round window, the oval window, and the cochlear aqueduct. The round window and oval window openings are protected by membranes which are used to facilitate pressure input and fluid expansion as auditory stimuli are transmitted as stapes-induced pressure waves in the cochlear perilymph. The round window membrane (RWM) opening provides an un-occluded entry point to the inner ear. Researchers and clinicians have utilized this access point as the route of delivery for therapeutics including cochlear implants and gene therapy vectors.

In the context of lipid and viral vector delivery for gene therapy, a large body of work has been generated to assess feasibility of many variations on this approach. A large number of viral vectors have been assessed in mouse and NHPs for their capacity to transduce cochlear cell types from the RWM point of entry. Non-invasive approaches have been attempted that avoid puncturing the RWM and instead rely on diffusion of vector particles across an intact or semi-intact membrane. Additional methods include canalostomy and cochleostomy which open new holes to access the cochlea [1]. Using these methods, a hole is opened directly into the semicircular canal and cochlea, respectively. Vector is then delivered through the created opening. Hybrid methods have also been utilized, for example a RWM+canalostomy approach has been developed, which leverages fluid flow, and efflux, to distribute vector particles throughout the entire cochlea [2]. All of the above approaches have been attempted with a wide assortment of vector types, including many canonical AAV capsid serotypes and more recently developed AAV capsid variants [3, 4].

The cochlear aqueduct has attracted relatively little attention as a possible route of AAV vector delivery. It is arguably less accessible, requiring access to the intracranial space. Furthermore, the volume of the CSF is much larger than that of the perilymph, and the brain occupying this space will take up vector reducing the viral load likely to reach the cochlea and posing safety risks greater than loss of hearing. However, if these risks could be mitigated by thoughtful therapeutic design, and if CSF based delivery was capable of robust cochlea transduction, CSF delivery could reduce surgical risks and unintended surgical damage to the cochlear tissues. Gene transfer via the CSF was first observed after administration of adenoviral vector to the contralateral ear in Guinea pigs [5]. In this study, systemic delivery of 25 μL of adenoviral vector was found to transduce both cochlea; however, transduction was limited to mesothelial cells near the opening of the cochlear aqueduct.

Non-traumatic, non-surgical methods for delivering molecular gene therapy tools to cochlear sensory and supporting cells are needed.

### SUMMARY

Provided herein are compositions and methods for transduction of a therapeutically effective number of sensory and supporting cells in both cochleae. These compositions and methods are grounded in the unexpected finding that multiple AAV vectors, when administered to the CSF, bilaterally transduced inner hair cells and supporting cells throughout all turns of the cochlea. These surprising findings demonstrate that robust gene transfer to the cochlea can be achieved via the CSF and cochlear aqueduct.

In one embodiment, provided herein are methods to deliver a therapeutic transgene to the cochlea of a subject, comprising administering to the cerebrospinal fluid of the subject a modified adeno-associated virus (AAV) encoding a therapeutic transgene. In some aspects, the therapeutic transgene treats or prevents a hearing or vestibular disorder when expressed in a cell of the cochlea. In some aspects, the modified AAV is of serotype AAV1, AAV2, AAV3, AAV4, AAV5, AAV6, AAV7, AAV8, AAV9, AAV10, AAV11, or AAV12.

In some aspects, the therapeutic transgene is operably linked to a promoter. In some aspects, the promoter is homologous to the therapeutic transgene, in other words, the promoter may be the promoter that is operably linked to the associated gene endogenously. In some aspects, the promoter is heterologous to the therapeutic transgene. In some aspects, the promoter is a cochlea-specific promoter. The cochlea-specific promoter may be a hair cell-specific promoter or a support cell-specific promoter (e.g., a GJB2 promoter).

In some aspects, the modified AAV comprises a modified capsid protein. The modified capsid protein may comprise a targeting peptide, which may be three to ten amino acids in length. In some aspects, the targeting peptide is seven amino acids in length. The modified AAV capsid protein may be a modified AAV1 capsid protein, a modified AAV2 capsid protein, or a modified AAV9 capsid protein.

In some aspects, the modified AAV capsid protein is derived from an AAV1 capsid protein (e.g., SEQ ID NO: 164), wherein the targeting peptide is inserted after residue 590 of the AAV1 capsid protein. In some aspects, the targeting peptide is flanked by linker sequences, wherein the linker sequences on each side of the targeting peptides are two or three amino acids long. In some aspects, the linker sequences are SSA on the N-terminal side of the targeting peptide and AS on the C-terminal side of the targeting peptide. In some aspects, the modified AAV1 capsid proteins have a sequence at least 95% identical to SEQ ID NO: 167. In some aspects, the targeting peptide is one of the peptides shown in FIG. 6. In some aspects, the targeting peptide is one of the peptides of SEQ ID NOs: 1-44, 150, and 151. In some aspects, the targeting peptide is one of the peptides of SEQ ID NOs: 39, 150, and 151. In some aspects, the targeting peptide is SEQ ID NO: 39. In some aspects, the targeting peptide is SEQ ID NO: 150. In some aspects, the targeting peptide is SEQ ID NO: 151.

In some aspects, the modified AAV capsid protein is derived from an AAV2 capsid protein (e.g., SEQ ID NO: 165), wherein the targeting peptide is inserted after residue 587 of the AAV2 capsid protein. In some aspects, the targeting peptide is flanked by linker sequences, wherein the linker sequences on each side of the targeting peptides are two or three amino acids long. In some aspects, the linker sequences are AAA on the N-terminal side of the targeting peptide and AA on the C-terminal side of the targeting peptide. In some aspects, the modified AAV2 capsid proteins have a sequence at least 95% identical to SEQ ID NO: 168. In some aspects, the targeting peptide is one of the peptides shown in FIG. 8. In some aspects, the targeting peptide is one of the peptides of SEQ ID NOs: 45-100, 152, and 154. In some aspects, the targeting peptide is one of the peptides of SEQ ID NOs: 84, 152, and 154. In some aspects, the targeting peptide is SEQ ID NO: 84. In some aspects, the targeting peptide is SEQ ID NO: 152. In some aspects, the targeting peptide is SEQ ID NO: 154.

In some aspects, the modified AAV capsid protein is derived from an AAV9 capsid protein (e.g., SEQ ID NO: 166), wherein the targeting peptide is inserted after residue 588 of the AAV9 capsid protein. In some aspects, the targeting peptide is flanked by linker sequences, wherein the linker sequences on each side of the targeting peptides are two or three amino acids long. In some aspects, the linker sequences are AAA on the N-terminal side of the targeting peptide and AS on the C-terminal side of the targeting peptide. In some aspects, the modified AAV9 capsid proteins have a sequence at least 95% identical to SEQ ID NO: 169. In some aspects, the targeting peptide is one of the peptides shown in FIG. 10. In some aspects, the targeting peptide is one of the peptides of SEQ ID NOs: 101-149, 153, and 155. In some aspects, the targeting peptide is SEQ ID NO: 153. In some aspects, the targeting peptide is SEQ ID NO: 155.

In certain embodiments, the viral vector is an adeno associated viral vector (AAV). In certain embodiments, the AAV is AAV1, AAV2, or AAV9. In certain aspects, a targeting peptide is inserted at position 590 of the AAV1 capsid, position 587 of the AAV2 capsid, or position 588 of the AAV9 capsid. In an exemplary modified AAV1 capsid protein, the targeting peptide insertion after position 590 as SSAX₇AS, where the leading SSA and the trailing AS are linker sequences and ×₇ represents the targeting peptide. In an exemplary modified AAV2 capsid protein sequence, the targeting peptide insertion after position 587 as AAAX-AA, where the leading AAA and the trailing AA are linker sequences and X₇ represents the targeting peptide. In an exemplary modified AAV9 capsid protein sequence, the targeting peptide insertion after position 588 as AAAX₇AS, where the leading AAA and the trailing AS are linker sequences and X₇ represents the targeting peptide.

In some aspects, the therapeutic transgene encodes an siRNA, shRNA, miRNA, non-coding RNA, lncRNA, therapeutic protein, or CRISPR system.

In some aspects, the administration is to a cisterna magna, an intraventricular space, a brain ventricle, a subarachnoid space, and/or an intrathecal space. In some aspects, the administration is through a intracerebroventricular delivery route. In some aspects, the delivery is to the cerebrospinal fluid. In some aspects, cochlear therapeutic delivery is facilitated by diffusion of therapeutic particles through the cochlear aqueduct to the perilymph filled scala vestibuli and scala tympani of the cochlea. In some aspects, the method delivers the therapeutic transgene to a cell of the inner ear. In some aspects, the cell in the inner ear is selected from the group consisting of spiral ganglion neurons, vestibular hair cells, vestibular ganglion neurons, supporting cells, and cells in the stria vascularis. In some aspects, the cell is a hair cell of the cochlea or vestibular system. In some aspects, the cell of the vestibular system is a hair cell of the utricle, or a cell in an ampulla of a lateral semicircular canal, or a hair cell in a cupula. In some aspects, the cell is an inner hair cell of the cochlea or an outer hair cell of the cochlea. In some aspects, the therapeutic transgene is delivered to at least 80% of inner hair cells and/or at least 80% of outer hair cells.

In some aspects, the subject has a hearing disorder, and the therapeutic transgene is delivered in a therapeutically effective amount. In some aspects, the subject is at risk of exposure to damaging auditory stimuli. In some aspects, the administering reverses or prevents hearing loss. In some aspects, the method treats hereditary hearing loss in the subject. In some aspects, the hearing loss is partial hearing loss or complete deafness.

In some aspects, the cell is a cell of the vestibular system, the subject has a disorder of the vestibular system, and the transgene is delivered in a therapeutically effective amount. In some aspects, the method treats or prevents impaired balance or impaired vestibular function in the subject.

In some aspects, a plurality of viral particles are administered. The virus are administered at a dose of about 1×10⁶ to about 1×10¹⁸ vector genomes per kilogram (vg/kg). In some aspects, the virus is administered at a dose from about 1×10⁷-1×1017, about 1×108-1×1016, about 1×109-1×1015, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-×10¹³, or about 1×10¹³-1×10¹⁴ vg/kg of the patient. In some aspects, the subject is human.

In one embodiment, provided herein is a modified adeno-associated virus (AAV) comprising a therapeutic transgene operably linked to a cochlea-specific promoter. In some aspects, the AAV is an AAV1, AAV2, AAV3, AAV4, AAV5, AAV6, AAV7, AAV8, AAV9, AAV10, AAV11, or AAV12. In some aspects, the therapeutic transgene treats or prevents a hearing or vestibular disorder.

In some aspects, the therapeutic transgene is operably linked to a promoter. In some aspects, the promoter is homologous to the therapeutic transgene, in other words, the promoter may be the promoter that is operably linked to the associated gene endogenously. In some aspects, the promoter is heterologous to the therapeutic transgene. In some aspects, the promoter is a cochlea-specific promoter. The cochlea-specific promoter may be a hair cell-specific promoter or a support cell-specific promoter (e.g., a GJB2 promoter).

In some aspects, the modified AAV comprises a modified capsid protein. The modified capsid protein may comprise a targeting peptide, which may be three to ten amino acids in length. In some aspects, the targeting peptide is seven amino acids in length. The modified AAV capsid protein may be a modified AAV1 capsid protein, a modified AAV2 capsid protein, or a modified AAV9 capsid protein.

In some aspects, the modified AAV capsid protein is derived from an AAV1 capsid protein (e.g., SEQ ID NO: 164), wherein the targeting peptide is inserted after residue 590 of the AAV1 capsid protein. In some aspects, the targeting peptide is flanked by linker sequences, wherein the linker sequences on each side of the targeting peptides are two or three amino acids long. In some aspects, the linker sequences are SSA on the N-terminal side of the targeting peptide and AS on the C-terminal side of the targeting peptide. In some aspects, the targeting peptide is one of the peptides shown in FIG. 6. In some aspects, the targeting peptide is one of the peptides of SEQ ID NOs: 1-44, 150, and 151. In some aspects, the targeting peptide is one of the peptides of SEQ ID NOs: 39, 150, and 151. In some aspects, the targeting peptide is SEQ ID NO: 39. In some aspects, the targeting peptide is SEQ ID NO: 150. In some aspects, the targeting peptide is SEQ ID NO: 151.

In some aspects, the modified AAV capsid protein is derived from an AAV2 capsid protein (e.g., SEQ ID NO: 165), wherein the targeting peptide is inserted after residue 587 of the AAV2 capsid protein. In some aspects, the targeting peptide is flanked by linker sequences, wherein the linker sequences on each side of the targeting peptides are two or three amino acids long. In some aspects, the linker sequences are AAA on the N-terminal side of the targeting peptide and AA on the C-terminal side of the targeting peptide. In some aspects, the targeting peptide is one of the peptides shown in FIG. 8. In some aspects, the targeting peptide is one of the peptides of SEQ ID NOs: 45-100, 152, and 154. In some aspects, the targeting peptide is one of the peptides of SEQ ID NOs: 84, 152, and 154. In some aspects, the targeting peptide is SEQ ID NO: 84. In some aspects, the targeting peptide is SEQ ID NO: 152. In some aspects, the targeting peptide is SEQ ID NO: 154.

In some aspects, the modified AAV capsid protein is derived from an AAV9 capsid protein (e.g., SEQ ID NO: 166), wherein the targeting peptide is inserted after residue 588 of the AAV9 capsid protein. In some aspects, the targeting peptide is flanked by linker sequences, wherein the linker sequences on each side of the targeting peptides are two or three amino acids long. In some aspects, the linker sequences are AAA on the N-terminal side of the targeting peptide and AS on the C-terminal side of the targeting peptide. In some aspects, the targeting peptide is one of the peptides shown in FIG. 10. In some aspects, the targeting peptide is one of the peptides of SEQ ID NOs: 101-149, 153, and 155. In some aspects, the targeting peptide is SEQ ID NO: 153. In some aspects, the targeting peptide is SEQ ID NO: 155.

In certain embodiments, the viral vector is an adeno associated viral vector (AAV). In certain embodiments, the AAV is AAV1, AAV2, AAV3, AAV4, AAV5, AAV6, AAV7, AAV8, AAV9, AAV10, AAV11, or AAV12. In certain aspects, a targeting peptide is inserted at position 590 of the AAV1 capsid, position 587 of the AAV2 capsid, or position 588 of the AAV9 capsid. In an exemplary modified AAV1 capsid protein, the targeting peptide insertion after position 590 as SSAX₇AS, where the leading SSA and the trailing AS are linker sequences and X₇ represents the targeting peptide. In an exemplary modified AAV2 capsid protein sequence, the targeting peptide insertion after position 587 as AAAX₇AA, where the leading AAA and the trailing AA are linker sequences and X₇ represents the targeting peptide. In an exemplary modified AAV9 capsid protein sequence, the targeting peptide insertion after position 588 as AAAX-AS, where the leading AAA and the trailing AS are linker sequences and X₇ represents the targeting peptide.

In some aspects, the therapeutic transgene encodes an siRNA, shRNA, miRNA, non-coding RNA, lncRNA, therapeutic protein, or CRISPR system.

In one embodiment, provided herein are pharmaceutical compositions comprising the modified AAV of any one of the present embodiments and a pharmaceutically acceptable carrier.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The temporal bone of a Rhesus macaque injected with intracerebroventricular (ICV) AAV9. Upon dissection of the temporal bone and removal of the bone covering the apical turn of the cochlea strong fluorescence emanated from transduced cochlear hair cell and supporting cells.

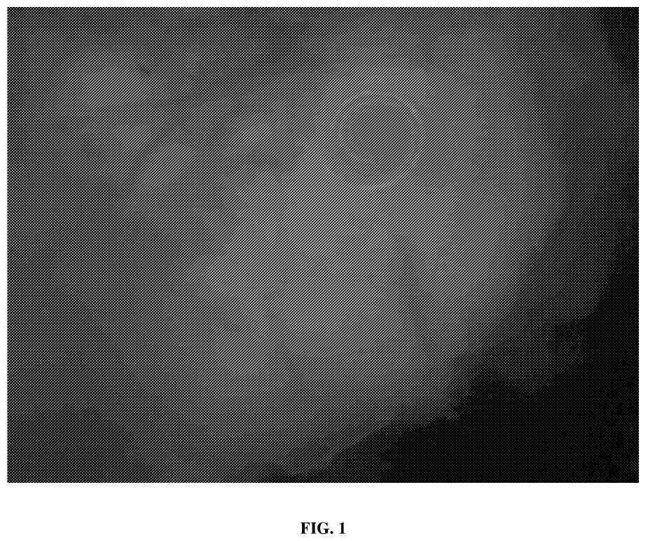

FIG. 2. Under an epifluorescence microscope tiled images were collected of a whole mount preparation of the Rhesus macaque cochlea from the ICV AAV9 injected animal. This preparation displays the organ of corti for the entire length of the cochlea from the apical to basal turns (apex, middle and base). Robust inner hair cell and supporting cell transduction was observed in the apical and middle turns while sparser IHC transduction was observed at the base. Cells transduced with AAV9 were observed throughout the turns of the cochlea suggesting that all tonotopic regions are targeted to some extent.

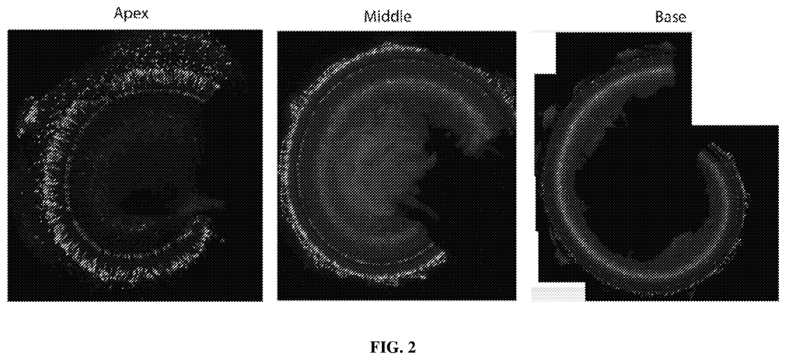

FIG. 3. Murine temporal bone from an animal injected ICV with an AAV2 variant. In this image the bone covering the apical turn of the cochlea has been removed. Transduced tissues include the facial nerve, genticulate ganglion and inner hair cells of the cochlea and the vestibular nerve.

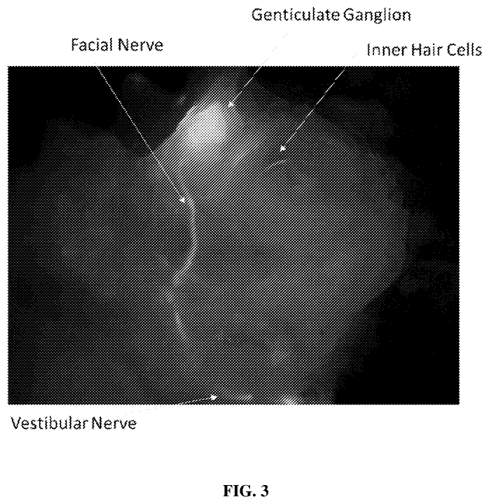

FIG. 4. Dissecting microscope images showing the extent and specificity of inner hair cell transduction across the apex and base of the mouse cochlea after ICV injection of an AAV2 variant.

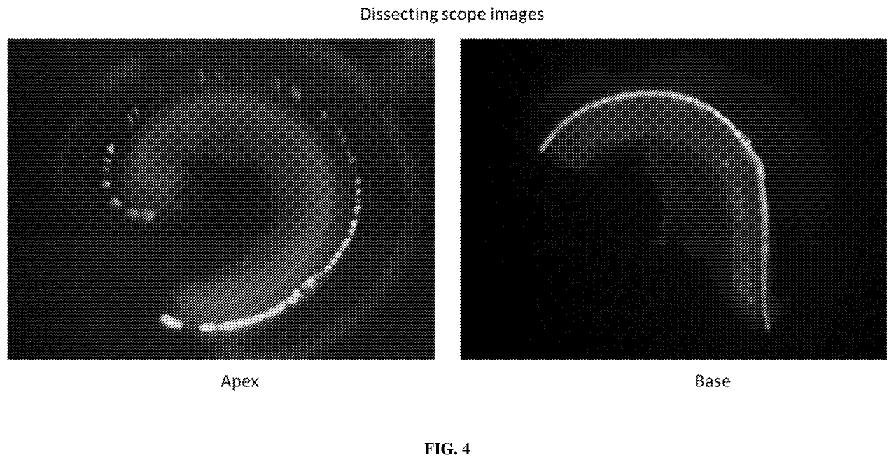

FIG. 5. Images showing inner hair cell (IHC) transduction in mouse cochlea transduced with an AAV2 variant that was delivered by Intracerebroventricular (ICV) injection. Remarkably strong and specific IHC transduction was observed to hit cells across the apex and base of the murine cochlea (which has one less turn than the Rhesus macaque). Here we show representative images of cochlear regions from the apex and base. Transduction was nearly >90% from the base of the cochlea through the apical middle. Interestingly at the very apical tip we observed less transduction, however this was limited to a very small area. This level of inner hair cell transduction is both therapeutically relevant and strikingly unexpected coming from an ICV brain injection into the CSF.

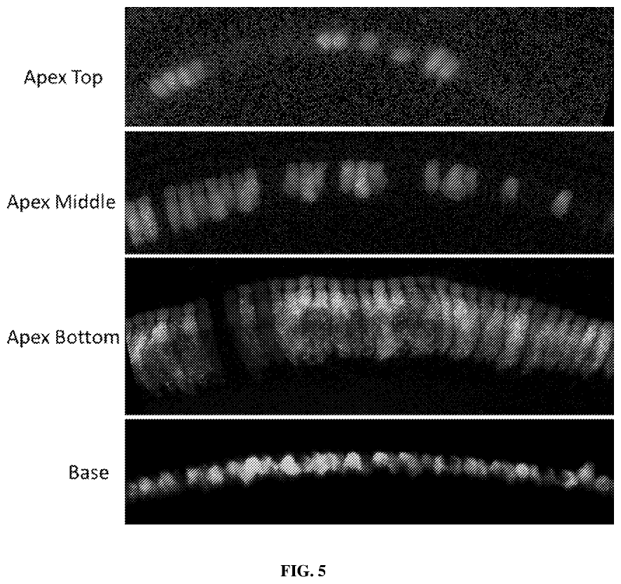

FIG. 6. Figures depicting the results from an AAV directed evolution experiment. A library of barcoded AAV1 variants was introduced into the cochlea by a direct cochlea injection delivered by RWM+canalostomy approach. Multiple rounds of injection were performed to enrich for functional capsids. Round1 indicates hits obtained from after the first round of in-vivo enrichment. Round2 (DNA and RNA) indicates hits obtained form after the second round of in-vivo enrichment. Several sequences were strongly enriched in cochlea tissues and/or vestibular tissues. The sequences shown for cochlea, from top to bottom, are SEQ ID NOs: 1-40. The sequences shown for vestibular tissues, from top to bottom, are SEQ ID NOs: 41, 6, 7, 3, 42, 43, 8, 12, 1, 5, 44, 4, 13, 19, 15-17, 11, 28, 20, 26, 18, 30, 25, 22, 23, 21, 31, 24, 29, 32, 40, 37, 27, 36, 34, 38, 33, 39, and 35. Capsid candidate LGGSAAR (SEQ ID NO: 39) was selected from among the AAV1 derived capsid candidates for its strong DNA and RNA performance in both Cochlea and pooled vestibular tissues.

FIG. 7. Correlation of AAV1 capsid variant detection of DNA and RNA hits between the cochlea and vestibular tissues. DNA capsid hits are strongly correlated between the two tissues while RNA hits are more variable with a lower number of highly correlated capsids.

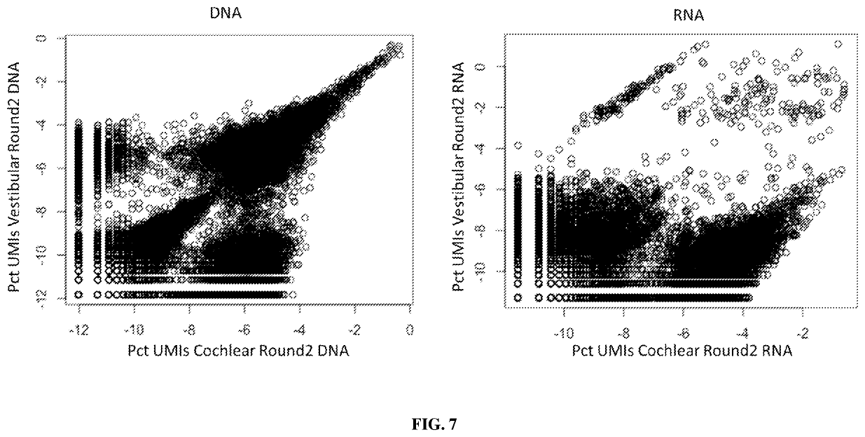

FIG. 8. Figures depicting the results from an AAV directed evolution experiment. A library of barcoded AAV2 variants was introduced into the cochlea by a direct cochlea injection delivered by RWM+canalostomy approach. Multiple rounds of injection were performed to enrich for functional capsids. Round1 indicates hits obtained from after the first round of in-vivo enrichment. Round2 (DNA and RNA) indicates hits obtained form after the second round of in-vivo enrichment. Several sequences were strongly enriched in cochlea tissues and/or vestibular tissues. The sequences shown for cochlea, from top to bottom, are SEQ ID NOs: 45-84. The sequences shown for vestibular tissues, from top to bottom, are SEQ ID NOs: 85, 86, 69, 87-91, 56, 92, 93, 62, 94, 66, 68, 95, 96, 73, 49, 51, 97-99, 70, 65, 47, 77, 71, 75, 76, 100, 78, 80, 63, 82, 79, 81, 72, 83, and 84. Capsid candidate KAGGSQG (SEQ ID NO: 84) was selected for its strong DNA and RNA performance in cochlea despite its reduced RNA performance in vestibular tissues.

FIG. 9. Correlation of AAV2 capsid variant detection of DNA and RNA hits between the cochlea and vestibular tissues. DNA capsid hits are strongly correlated between the two tissues while RNA hits are more variable with a lower number of highly correlated capsids.

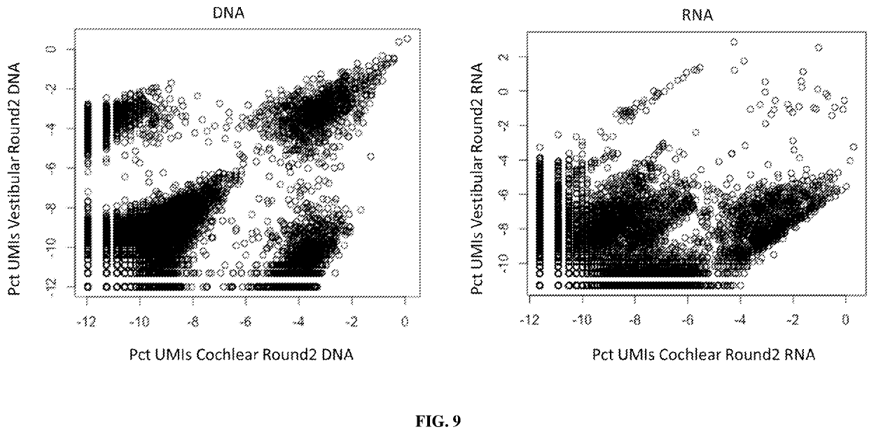

FIG. 10. Figures depicting the results from an AAV directed evolution experiment. A library of barcoded AAV9 variants was introduced into the cochlea by a direct cochlea injection delivered by RWM+canalostomy approach. Multiple rounds of injection were performed to enrich for functional capsids. Round1 indicates hits obtained from after the first round of in-vivo enrichment. Round2 (DNA and RNA) indicates hits obtained form after the second round of in-vivo enrichment. Several sequences were strongly enriched in cochlea tissues and/or vestibular tissues. The sequences shown for cochlea, from top to bottom, are SEQ ID NOs: 101-140. The sequences shown for vestibular tissues, from top to bottom, are SEQ ID NOs: 110, 141-143, 117, 105, 144, 145, 129, 146, 147, 109, 130, 115, 114, 148, 111, 128, 113, 104, 121, 131, 102, 120, 149, 126, 127, 122, 125, 123, 124, 134, 133, 135, 132, 137, 136, and 138-140. No capsid candidates were selected from AAV9 as a limited number of fluorescence validation positions (8) are available and other criteria were also considered.

FIG. 11. Correlation of AAV9 capsid variant detection of DNA and RNA hits between the cochlea and vestibular tissues. DNA capsid hits are strongly correlated between the two tissues while RNA hits are more variable with a lower number of highly correlated capsids.

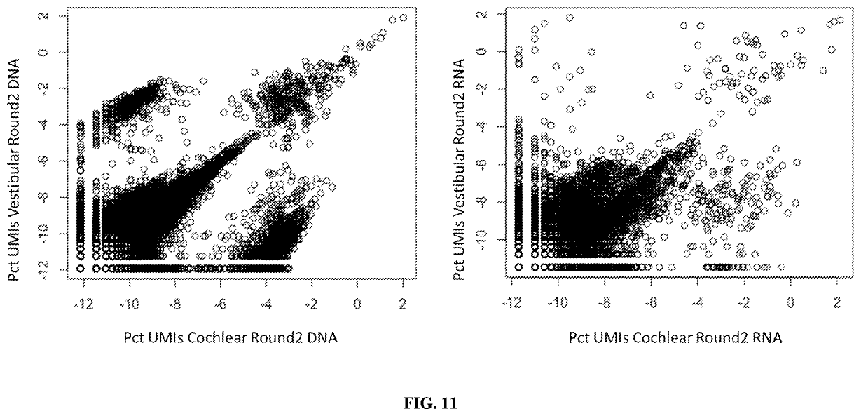

FIG. 12. Rankings of capsid candidates by average round2 DNA UMI counts and average fold enrichment of round2 DNA UMI counts over UMI counts from input virus. Additional capsid candidates were selected from rankings of the average UMI counts as well as the fold enrichment of % Round 2 DNA UMI counts over % DNA UMI counts obtained from the Round2 Input Vector Pool. The top non-normalized hit sequence for AAV1 was LGGSAAR (SEQ ID NO: 39) and for AAV2 was KAGGSQG (SEQ ID NO: 84). The top normalized hits for AAV1 were IDVGSAD (SEQ ID NO: 150) and FAAMGSL (SEQ ID NO: 151), for AAV2 was PPYAMVM (SEQ ID NO: 152), and for AAV9 was SRGSGPS (SEQ ID NO: 153).

FIGS. 13A-C. Selection of intermediate capsid candidates with strong detection to input ratios. FIG. 13A. No capsid candidates were selected using this method from AAV1. FIG. 13B. AAV2 derived candidate AAKVAAP (SEQ ID NO: 154) was selected using this method. FIG. 13C. AAV9 derived candidate RSGVGSA (SEQ ID NO: 155) was selected using this method.

FIGS. 14A-C. Capsid candidates selected for in vivo fluorescence validation. FIG. 14A. All capsid candidates selected to be carried forward into fluorescence validation are listed (from top to bottom: SEQ ID NOs: 39, 84, and 150-155) with their indicated parental serotype and a description of the criteria used to select that capsid. FIG. 14B. To facilitate delivery of separately fluorescently tagged capsid, one into each ear of a non-human primate, capsids were grouped into two validation pools. Pool 1 peptide sequences, from top to bottom: SEQ ID NO: 39, 84, 150, and 152. Pool 1 DNA sequences, from top to bottom: SEQ ID NOs: 156-159. Pool 2 peptide sequences, from top to bottom: SEQ ID NO: 154, 155, 151, and 153. Pool 2 DNA sequences, from top to bottom: SEQ ID NOs: 160-163. FIG. 14C. Capsid candidates were individually generated to deliver a fluorescence reporter expression construct. Capsids were pooled in groups of 4, such that the above validation pools were created. One validation pool will be delivered by direct intra-cochlear injection to each inner ear of a Rhesus Macaque. Thirty days post injection the animal will be sacrificed, and cochlea collected for histological evaluation.

### DETAILED DESCRIPTION

Provided herein are unexpected findings that a single intracerebroventricular injection of AAV vector into the CSF is sufficient to transduce a therapeutically relevant number of sensory and supporting cells in both cochleae. Surprisingly, robust and extensive transduction of the cochlea was observed in the Rhesus macaque. AAV9, when delivered by ICV injection into the CSF, bilaterally transduced inner hair cells and supporting cells throughout all turns of the cochlea. Other vectors delivered ICV in mice were found to have similar cochlear transduction capacity with varying cell type specificity. An AAV2-derived capsid variant delivered ICV to mice was found to be highly specific for inner hair cells, yielding almost complete IHC transduction in all turns but the apical tip. These surprising findings reveal that robust gene transfer to the cochlea can be achieved via the CSF and cochlear aqueduct. The observation of this gene transfer in adult Rhesus macaques suggests that a similar ICV based approach is likely to be successful in humans.

In certain embodiments, the AAV is AAV1, AAV2, or AAV9. An exemplary wildtype reference AAV1 capsid protein sequence is provided in SEQ ID NO: 164. An exemplary wildtype reference AAV2 capsid protein sequence is provided in SEQ ID NO: 165. An exemplary wildtype reference AAV9 capsid protein sequence is provided in SEQ ID NO: 166. In certain aspects, the targeting peptide is inserted at position 590 of the AAV1 capsid, position 587 of the AAV2 capsid, or position 588 of the AAV9 capsid. An exemplary modified AAV1 capsid protein sequence is provided in SEQ ID NO: 167, which shows the targeting peptide insertion after position 590 as SSAX₇AS, where the leading SSA and the trailing AS are linker sequences and X₇ represents the targeting peptide. An exemplary modified AAV2 capsid protein sequence is provided in SEQ ID NO: 168, which shows the targeting peptide insertion after position 587 as AAAX-AA, where the leading AAA and the trailing AA are linker sequences and X₇ represents the targeting peptide. An exemplary modified AAV9 capsid protein sequence is provided in SEQ ID NO: 169, which shows the targeting peptide insertion after position 588 as AAAX-AS, where the leading AAA and the trailing AS are linker sequences and X₇ represents the targeting peptide.

### I. ADENO-ASSOCIATED VIRUS (AAV) VECTORS

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for plasmid propagation and production, but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genome of most native AAVs often contain two open reading frames (ORFs), sometimes referred to as a left ORF and a right ORF. The right ORF often encodes the capsid proteins VP1, VP2, and VP3. These proteins are often found in a ratio of 1:1:10 respectively, but may be in varied ratios, and are all derived from the right-hand ORF. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles. In certain embodiments, the genome of an AAV particle encodes one, two or all three VP1, VP2 and VP3 polypeptides.

The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The term “recombinant,” as a modifier of vector, such as recombinant viral, e.g., lenti- or parvo-virus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild type genome of a virus by using molecular methods to remove part of the wild type genome from the virus, and replacing with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-218.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV2 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-218 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-218 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-218 serotype.

In certain embodiments, a method herein comprises use, administration or delivery of an rAAV1, rAAV2, rAAV3, rAAV4, rAAV5, rAAV6, rAAV7, rAAV8, rAAV9, rAAV10, rAAV11, rAAV12, rRh10, rRh74 or rAAV-218 particle.

In certain embodiments, a method herein comprises use, administration or delivery of a rAAV2 particle. In certain embodiments a rAAV2 particle comprises an AAV2 capsid. In certain embodiments a rAAV2 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments a rAAV2 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV2 particle. In certain embodiments, a rAAV2 particle is a variant of a native or wild-type AAV2 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV2 particle.

In certain embodiments a rAAV9 particle comprises an AAV9 capsid. In certain embodiments a rAAV9 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments a rAAV9 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV9 particle. In certain embodiments, a rAAV9 particle is a variant of a native or wild-type AAV9 particle. In some aspects, one or more capsid proteins of an AAV9 variant have 1, 2, 3, 4, 5, 5-10, 10-15, 15-20 or more amino acid substitutions compared to capsid protein(s) of a native or wild-type AAV9 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-218, as long as they retain one or more desired ITR functions (e.g., ability to form a hairpin, which allows DNA replication; integration of the AAV DNA into a host cell genome; and/or packaging, if desired).

In certain embodiments, a rAAV2 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

In certain embodiments, a rAAV9 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV2 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction-competent AAV particles and propagated using an AAV viral packaging system. A transduction-competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent, and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction-competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral E1a and E1b genes is used to generate recombinant AAV particles. The modified HEK293 cell line is readily transfected, and provides a particularly convenient platform in which to produce rAAV particles. Methods of generating high titer AAV particles capable of transducing mammalian cells are known in the art. For example, AAV particle can be made as set forth in Wright, 2008 and Wright, 2009.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

An “expression vector” is a specialized vector that contains a gene or nucleic acid sequence with the necessary regulatory regions needed for expression in a host cell. An expression vector may contain at least an origin of replication for propagation in a cell and optionally additional elements, such as a heterologous nucleic acid sequence, expression control element (e.g., a promoter, enhancer), intron, ITR(s), and polyadenylation signal.

### II. THERAPEUTIC AGENTS

In some embodiments, viral gene transfer methods can be used to introduce nucleic acids in mammalian cells or target tissues. Such methods can be used to administer nucleic acids encoding inhibitory RNAs, non-coding RNAs, and/or therapeutic proteins to cells in culture or in a host organism.

### A. Inhibitory RNAs

“RNA interference (RNAi)” is the process of sequence-specific, post-transcriptional gene silencing initiated by siRNA. During RNAi, siRNA induces degradation of target mRNA with consequent sequence-specific inhibition of gene expression.

An “inhibitory RNA,” “RNAi,” “small interfering RNA” or “short interfering RNA” or “siRNA” molecule, “short hairpin RNA” or “shRNA” molecule, or “miRNA” is an RNA duplex of nucleotides that is targeted to a nucleic acid sequence of interest. As used herein, the term “siRNA” is a generic term that encompasses the subset of shRNAs and miRNAs. An “RNA duplex” refers to the structure formed by the complementary pairing between two regions of an RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding huntingtin. In some embodiments, the length of the duplex of siRNAs is less than 30 base pairs. In some embodiments, the duplex can be 29, 28, 27, 26, 25, 24, 23, 22, 21, 20, 19, 18, 17, 16, 15, 14, 13, 12, 11 or 10 base pairs in length. In some embodiments, the length of the duplex is 19 to 25 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24 or 25 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

shRNAs are comprised of stem-loop structures which are designed to contain a 5′ flanking region, siRNA region segments, a loop region, a 3′ siRNA region and a 3′ flanking region. Most RNAi expression strategies have utilized short-hairpin RNAs (shRNAs) driven by strong polIII-based promoters. Many shRNAs have demonstrated effective knock down of the target sequences in vitro as well as in vivo, however, some shRNAs which demonstrated effective knock down of the target gene were also found to have toxicity in vivo.

miRNAs are small cellular RNAs (˜22 nt) that are processed from precursor stem loop transcripts. Known miRNA stem loops can be modified to contain RNAi sequences specific for genes of interest. miRNA molecules can be preferable over shRNA molecules because miRNAs are endogenously expressed. Therefore, miRNA molecules are unlikely to induce dsRNA-responsive interferon pathways, they are processed more efficiently than shRNAs, and they have been shown to silence 80% more effectively.

A recently discovered alternative approach is the use of artificial miRNAs (pri-miRNA scaffolds shuttling siRNA sequences) as RNAi vectors. Artificial miRNAs more naturally resemble endogenous RNAi substrates and are more amenable to Pol-II transcription (e.g., allowing tissue-specific expression of RNAi) and polycistronic strategies (e.g., allowing delivery of multiple siRNA sequences). See U.S. Pat. No. 10,093,927, which is incorporated by reference.

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional siRNAs. “Artificial miRNA” or an “artificial miRNA shuttle vector”, as used herein interchangeably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A siRNA target generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. Any gene being expressed in a cell can be targeted. Preferably, a target gene is one involved in or associated with the progression of cellular activities important to disease or of particular interest as a research object.

### B. Non-Coding RNAs

As evidenced by cDNA cloning projects and genomic tiling arrays, more than 90% of the human genome undergoes transcription but does not code for proteins. These transcriptional products are referred to as non-protein coding RNAs (ncRNAs). A variety of ncRNA transcripts, such as ribosomal RNAs, transfer RNAs, competing endogenous RNA (ceRNA), small nuclear RNA (snRNA), and small nucleolar RNA (snoRNA), are essential for cell function. Similarly, a large number of short ncRNAs such as micro-RNAs (miRNAs), endogenous short interfering RNAs (siRNAs), PIWI-interacting RNAs (piRNAs), and small nucleolar RNAs (snoRNAs) are also known to play important regulatory roles in eukaryotic cells. Recent studies have demonstrated a group of long ncRNA (lncRNA) transcripts that exhibit cell type-specific expression and localize into specific subcellular compartments. lncRNAs are also known to play important roles during cellular development and differentiation supporting the view that they have been selected during the evolutionary process.

LncRNAs appear to have many different functions. In many cases, they seem to play a role in regulating the activity or localization of proteins, or serve as organizational frameworks for subcellular structures. In other cases, lncRNAs are processed to yield multiple small RNAs or they may modulate how other RNAs are processed. The latest edition of data produced by the public research consortium GenCode (version #27) catalogs just under 16,000 lncRNAs in the human genome, producing nearly 28,000 transcripts; when other databases are included, more than 40,000 lncRNAs are known.

Interestingly, lncRNAs can influence the expression of specific target proteins at specific genomic loci, modulate the activity of protein binding partners, direct chromatin-modifying complexes to their sites of action, and are post-transcriptionally processed to produce numerous 5′-capped small RNAs. Epigenetic pathways can also regulate the differential expression of lncRNAs.

A growing body of evidence also suggests that aberrantly expressed lncRNAs play important roles in normal physiological processes as well as multiple disease states. lncRNAs are misregulated in various diseases, including ischaemia, heart disease, Alzheimer's disease, psoriasis, and spinocerebellar ataxia type 8. This misregulation has also been shown in various types of cancers, such as breast cancer, colon cancer, prostate cancer, hepatocellular carcinoma and leukemia. Several lncRNAs, e.g. gadd74 and lncRNA-RoR5, modulate cell cycle regulators such as cyclins, cyclin-dependent kinases (CDKs), CDK inhibitors and p53 and thus provide an additional layer of flexibility and robustness to cell cycle progression. In addition, some lncRNAs are linked to mitotic processes such as centromeric satellite RNA, which is essential for kinetochore formation and thus crucial for chromosome segregation during mitosis in humans and flies. Another nuclear lncRNA, MA-lincl, regulates M phase exit by functioning in cis to repress the expression of its neighbouring gene Pura, a regulator of cell proliferation.

lncRNAs are a group that is commonly defined as transcripts of more than 200 nucleotides (e.g. about 200 to about 1200 nt, about 2500 nt, or more) that lack an extended open reading frame (ORF). The term “non-coding RNA” (ncRNA) includes lncRNA as well as shorter transcripts of, e.g., less than about 200 nt, such as about 30 to 200 nt.

Thus, in some embodiments, delivery of a ncRNA, such as to a specific brain structure of interest, corrects aberrant RNA expression levels or modulates levels of disease-causing lncRNA. Accordingly, in some embodiments, the present invention provides an rAAV, wherein the viral genome is engineered to encode a therapeutic non-coding RNA (ncRNA). In some embodiments, the ncRNA is a long non-coding RNA (lncRNA) of about 200 nucleotides (nt) in length or greater. In some embodiments, the therapeutic is a ncRNA of about 25 nt or about 30 nt to about 200 nt in length. In some embodiments, the lncRNA is about 200 nt to about 1,200 nt in length. In some embodiments, the lncRNA is about 200 nt to about 1,100, about 1,000, about 900, about 800, about 700, about 600, about 500, about 400, or about 300 nt in length.

### C. CRISPR Systems

Gene editing is a technology that allows for the modification of target genes within living cells. Recently, harnessing the bacterial immune system of CRISPR to perform on demand gene editing revolutionized the way scientists approach genomic editing. The Cas9 protein of the CRISPR system, which is an RNA guided DNA endonuclease, can be engineered to target new sites with relative ease by altering its guide RNA sequence. This discovery has made sequence specific gene editing functionally effective.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor (e.g., KRAB) or activator, to affect gene expression. Alternatively, a CRISPR system with a catalytically inactivate Cas9 further comprises a transcriptional repressor or activator fused to a ribosomal binding protein.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. The Cas enzyme may be a target gene under the control of a regulated alternative splicing event, as disclosed herein, either as a chimeric target gene minigene or as a target gene for a chimeric minigene transactivator. The gRNA may be under the control of a constitutive promoter.

Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### D. Therapeutic Proteins

Some embodiments concern expression of recombinant proteins and polypeptides. Such proteins may be otoprotective proteins, such as an anti-apoptotic protein, an anti-oxidant enzyme (e.g., those belonging to the superoxide dismutase (SOD) family), a neurotrophic/neuroprotective factor, an anti-inflammatory protein, or a protein that promoters hair cells regeneration in the vestibular system. The therapeutic protein may be Birc1a (NAIP), Birc2 (c-IAP1/HIAP-2), Birc3 (cIAP-2/HIAP-1), Birc4 (XIAP), Birc5 (survivin), Birc6 (apollon), Birc7 (livin), Birc8 (TsIAP); members of the Bcl-2 family: Bcl-2, Bcl-XL, Bcl-w, Mcl-1, Bcl-2L10, BFL-1; endogenous inhibitors of the c-Jun N-terminus kinase (JNK) known as Jun-interacting protein (JIP), JIP-1, JIP-2, JIP-3, JIP-4; SOD1, SOD2; catalase; peroxiredoxin-1, peroxiredoxin-2, glutathione preoxidase 1 (Gpx1), Gpx2, Gpx3, or Gpx4; NGF, BDNF, CNTF, GDNF, Growth/differentiation factor-15 (GDF-15), erythropoietin or vascular endothelial growth factor (VEGF); interleukin-10 (IL-10); glutathione S-transferase, Annexin-1 (ANXA1), inhibitor of NF-κB (IκB); USH1, USHIG (a.k.a. SANS), CIB2 (calcium and integrin binding protein 2), ATOH-1, ACTG1, ATP2B2, CDH23 (cadherin 23), CLDN14, CLRN1, COCH, COL11A2, DFNA5, DFNB31, DFNB59, ESPN, EYA4, GJB2, GJB3, GJB6, KCNQ4, LHFPL5, MT-RNR1, MT-TS1, MYOIA, MY06, MYO7A (myosin 7a), MYO15A, OTOF, PCDH15 (protocadherin 15), PDZD7, POU3F4, SLC26A4, STRC, TECTA, TMC1, TMC2, TMIE, TMPRSS3, TRIOBP, USHIC, VLGR1, WFS1, ACTG1, ADCY1, ATOFfl, ATP6V1B1, BDNF, BDP1, BSND, DATSPER2, CABP2, CD 164, CDC 14 A, CDH23, CEACAM16, CHD7, CCDC50, CIB2, CLDN14, CLIC5, CLPP, CLRN1, COCH, COL2A1, COL4A3, COL4A4, COL4A5, COL9A 1, COL9A2, COLI 1 A1, COL1 1 A2, CRYM, DCDC2, DFNA5, DFNB31, DFNB59, DIAPH1, EDN3, EDNRB, ELMOD3, EMOD3, EPS8, EPS8L2, ESPN, ESRRB, EYA1, EYA4, FAM65B, FOXI1, GIPC3, GJB2, GJB3, GJB6, GPR98, GRHL2, GPSM2, GRXCR1, GRXCR2, HARS2, HGF, HOMER2, HSD17B4, ILDR1, KARS, KCNE1, KCNJ10, KCNQ1, KCNQ4, KITLG, LARS2, LHFPL5, LOXHD1, LRTOMT, MARVELD2, MCM2, MET, MIR 183, MIRN96, MITF, MSRB3, MT-RNR1, MT-TS 1, MYH14, MY′H9, MY015A, MYOIA, MY03A, MY06, MY07A, NARS2, NDP, NF2, NT3, OSBPL2, OTOA, OTOF, OTOG, OTOGL, P2RX2, PAX3, PCDH15, PDZD7, PJVK, PNPTI, POLRID, POLRIC, POU3F4, POU4F3, PRPS 1, PTPRQ, RDX, SIPR2, SANS, SEMA3E, SERPINB6, SLC17A8, SLC22A4, SLC26A4, SLC26A5, SIX1, SIX5, S MAC/DIABLO, SNAI2, SOX 10, STRC, SYNE4, TBC 1 D24, TCOF1, TECTA, TIMM8A, TJP2, TNC, TMC1, TMC2, TMIE, TMEM 132E, TMPRSS3, TRPN, TRIOBP, TSPEAR, USH1 C, USH1 G, USH2A, USH2D, VLGR1, WFS1, WHRN, or XIAP. Further exemplary therapeutic proteins and the associated conditions are listed in Table A.

TABLE A

| | | NCBI RefSeq ID | | |
|---|---|---|---|---|
| Disease or condition | Gene | GENE | Protein | C/V |
| Autosomal Recessive | TMIE | NG_011628.1 | NP_671729.2 | C |
| Nonsyndromic Hearing Loss | | | | |
| Autosomal Recessive | TMC1 | NG_008213.1 | NP_619636.2 | C |
| Nonsyndromic Hearing Loss | | | | |
| Sensorineural hearing loss | ATOH1 | NC_000004.12 | NP_005163.1 | C |
| Autosomal Recessive | Lhfp15 | NG_012184.1 | NP_872354.1 | CN |
| Nonsyndromic Hearing Loss | | | | |
| Usher syndrome type 3a | C1rn1 | NG_009168.1 | NP_777367.1 | C |
| Usher syndrome type 1c | Ush1C | NG_011883.1 | NP_001284693.1 | CN |
| | (harmonin) | | | |
| Waardenburg syndrome I, III | PAX3 | NG_011632.1 | NP_852122.1 | C |
| Waardenburg syndrome II | MITF | NG_011631.1 | NP_000239.1 | C |
| Waardenburg syndrome IV | EDNRB | NG_011630.2 | NP_000106.1 | C |
| | EDN3 | NG_008050.1 | NP_996917.1 | |
| | SOX10 | NG_007948.1 | NP_008872.1 | |
| Pendred syndrome | SLC26A4 | NG_008489.1 | NP_000432.1 | C |
| | (Pendrin) | | | |
| Episodic Ataxia 1 | KCNA1 | NG_011815.1 | NP_000208.2 | V |
| Episodic Ataxia 2 | CACNA1A | NG_011569.1 | NP_000059.3 | V |
| Episodic Ataxia 5 | CACNB4 | NG_012641.1 | NP_001005747.1 | V |
| Episodic Ataxia 6 | SLC1A3 | NG_015890.1 | NP_004163.3 | V |
| Various forms of autosomal | CX26/GJB2 | NG_008358.1 | NP_003995.2 | C |
| recessive deafness | | | | |

C: cochlea-hearing disorder,
V: vestibular system disorder

Additional conditions and the associated genetic defects have been described in the art; see, e.g., Kemperman et al., J R Soc Med. 2002 April; 95(4): 171-177; Gazquez and Lopez-Escamez, Curr Genomics. 2011 September; 12(6): 443-450; Duan et al., Gene Therapy (2004) 11:$51-S56.

When the present application refers to the function or activity of “modified protein” or a “modified polypeptide,” one of ordinary skill in the art would understand that this includes, for example, a protein or polypeptide that possesses an additional advantage over the unmodified protein or polypeptide. It is specifically contemplated that embodiments concerning a “modified protein” may be implemented with respect to a “modified polypeptide,” and vice versa.

Recombinant proteins may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein, but may possess the specificity and/or activity of the native protein. A “modified deleted protein” may also have reduced immunogenicity or antigenicity. An example of a modified deleted protein is one that has an amino acid residue deleted from at least one antigenic region, i.e. a region of the protein determined to be antigenic in a particular organism, such as the organism to which the modified protein is being administered.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue. Terminal additions, called fusion proteins, are discussed below.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

As used herein, a protein or peptide generally refers, but is not limited to, a protein of greater than about 200 amino acids, up to a full-length sequence translated from a gene; a polypeptide of greater than about 100 amino acids; and/or a peptide of from about 3 to about 100 amino acids. For convenience, the terms “protein,” “polypeptide,” and “peptide are used interchangeably herein.

As used herein, an “amino acid residue” refers to any naturally occurring amino acid, any amino acid derivative, or any amino acid mimic known in the art. In certain embodiments, the residues of the protein or peptide are sequential, without any non-amino acids interrupting the sequence of amino acid residues. In other embodiments, the sequence may comprise one or more non-amino acid moieties. In particular embodiments, the sequence of residues of the protein or peptide may be interrupted by one or more non-amino acid moieties.

Accordingly, the term “protein or peptide” encompasses amino acid sequences comprising at least one of the 20 common amino acids found in naturally occurring proteins, or at least one modified or unusual amino acid.

Certain embodiments of the present invention concern fusion proteins. These molecules may have a therapeutic protein linked at the N- or C-terminus to a heterologous domain. For example, fusions may also employ leader sequences from other species to permit the recombinant expression of a protein in a heterologous host. Another useful fusion includes the addition of a protein affinity tag, such as a serum albumin affinity tag or six histidine residues, or an immunologically active domain, such as an antibody epitope, preferably cleavable, to facilitate purification of the fusion protein. Non-limiting affinity tags include polyhistidine, chitin binding protein (CBP), maltose binding protein (MBP), and glutathione-S-transferase (GST).

Methods of generating fusion proteins are well known to those of skill in the art. Such proteins can be produced, for example, by de novo synthesis of the complete fusion protein, or by attachment of the DNA sequence encoding the heterologous domain, followed by expression of the intact fusion protein.

Production of fusion proteins that recover the functional activities of the parent proteins may be facilitated by connecting genes with a bridging DNA segment encoding a peptide linker that is spliced between the polypeptides connected in tandem. The linker would be of sufficient length to allow proper folding of the resulting fusion protein.

Expression of a transgene may be directed by the transgene's natural promoter (i.e., the promoter found naturally with the transgenic coding sequence) or expression of a transgene may be directed by a heterologous promoter (e.g., CMV promoter, Espin promoter, a PCDH15 promoter, a PTPRQ promoter and a TMHS (LHFPL5) promoter). For example, any of the transgenes described herein can be used with its natural promoter. Alternatively, any of the transgenes described herein can be used with a heterologous promoter. As used herein, a heterologous promoter refers to a promoter that does not naturally direct expression of that sequence (i.e., is not found with that sequence in nature). Representative heterologous promoters that can be used to direct expression of any of the transgenes indicated herein include, for example, a CMV promoter, a CBA promoter, a CASI promoter, a P promoter, and a EF-1 promoter, an alpha9 nicotinic receptor promoter, a prestin promoter, a Gfil promoter, and a Vglut3 promoter. In addition, a promoter that naturally directs expression of one of the above-referenced transgenes (e.g., a KCNQ4 promoter, a Myo7a promoter, a Myo6 promoter or an Atohl promoter) can be used as a heterologous promoter to direct expression of a transgene. In other embodiments, the promoter is an Espin promoter, a PCDH15 promoter, a PTPRQ promoter and a TMHS (LHFPL5) promoter.

### III. METHODS OF TREATMENT AND ADMINISTRATION

Viral vectors in some aspects may be administered directly to patients (in vivo) or they can be used to treat cells in vitro or ex vivo, and then administered to patients. In particular, provided herein are methods for inducing expression of a transgene in a cell of the inner ear. In some embodiments, the cell is a hair cell of the cochlea or vestibular system. In some embodiments, the cell is an inner hair cell of the cochlea or an outer hair cell of the cochlea; in some of these embodiments, the subject has a hearing disorder, and the transgene is delivered in a therapeutically effective amount. In some embodiments, the AAV vector transduces at least about 70% of cells of the inner ear; the AVV targets inner and outer hair cells with at least about 70%, 80%, 90%, 95% or greater efficiency, even as high as 100% efficiency. In some embodiments, the cell is a cell of the vestibular system, e.g., a hair cell of the utricle, or a cell in an ampulla of a lateral semicircular canal, or a hair cell in a cupula. In some embodiments wherein the cell is a cell of the vestibular system, the subject has a disorder of the vestibular system, and the transgene is delivered in a therapeutically effective amount.

The cochlea has two types of hair cells: Inner hair cells (IHCs) convert the mechanical stimulus of sound vibration into a neural signal transmitted by type I spiral ganglion neurons to the brain. Outer hair cells (OHCs) connect only to poorly defined type II neurons; their main function is to amplify the vibration produced by sound by as much as 60 decibels (dB) in a frequency-specific manner, and they are essential for frequency discrimination (important in speech perception). Most deafness genes known to affect hair cell function are expressed in both cell types, so in general, a useful gene therapy strategy should target both IHCs and OHCs. Cells, including hair cells, in the vestibular system, e.g., in the semicircular ducts (horizontal, anterior and posterior) or two otolith organs (saccule and utricle), are essential for our sense of balance and for coordinating eye movements; they are often affected in hereditary deafness so gene therapies should target them as well.

The term “vector” refers to small carrier nucleic acid molecule, a plasmid, virus (e.g., AAV vector, retroviral vector, lentiviral vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

These compositions can be used to treat a condition associated with loss of hearing or vestibular dysfunction, wherein the condition is caused by a genetic defect or is ameliorated by genetic therapy. Thus, in some embodiments, the methods described herein are used to treat a condition listed in Table A, using the corresponding sequence listing in Table A, in a subject in need thereof. Examples include certain forms of Usher syndrome (deafness associated with blindness and in some forms vestibular dysfunction).

In one example, the hearing loss is presbycusis. In another example, the hearing loss is high-frequency hearing loss. The high-frequency hearing loss is at 2 kHz and above. In yet another example, the hearing loss is due to ototoxicity, noise induced hearing loss, viral infections of the inner ear, autoimmune inner ear diseases, genetic hearing losses, inner ear barotrauma; physical trauma, or surgical trauma; or inflammation. The ototoxicity results from cisplatin treatment of the subject suffering from cancer. In one example, the hereditary hearing loss is Usher's I syndrome, Usher's II syndrome or Usher's III syndrome. In one example, the impaired balance is in a subject who is aging. In another example, the impaired vestibular function is result of vestibular organ degeneration. The vestibular organ regeneration is due to ototoxicity, viral infections of the inner ear, autoimmune inner ear diseases, genetic vestibular losses, inner ear barotraumas; or physical trauma, or surgical trauma.

For example, genetically based hearing loss is a significant problem with few therapeutic options other than cochlear implants. Inherited hearing problems are often due to single gene defects. Prelingual deafness is diagnosed in 1/500 infants, of which about 50% have a genetic etiology. Usher syndrome, which is associated with a number of different clinical subtypes, each of which can be caused by a mutation in any of a number of different genes, is responsible for 3 to 6% of early childhood deafness. One of the more prevalent genetic defects, estimated to be 1-2% of all genetic deafness, occurs in the TMC1 gene. Usher syndrome is classified under three clinical subtypes (USH-1, -2 and -3) according to the severity of the symptoms. USH1 is the most severe form. Patients who are affected by USH1 suffer congenital bilateral profound sensorineural hearing loss, vestibular areflexia and pre-pubertal retinitis pigmentosa (a progressive, bilateral, symmetric degeneration of rod and cone function of the retina). Unless fitted with a cochlear implant, individuals do not typically develop the ability to generate speech. While no biological treatments currently exist for Usher patients, early re introduction of the wild-type form of the defective gene may allow for reversal of the disease.

The most severe form of Usher Syndrome, USH1, is associated with defects in six genes: USH1, MY07A (myosin 7a), USHIC (harmonin), CDH23(cadherin 23), PCDH15 (protocadherin 15), SANS (sans; also known as USHIG) and CIB2 (calcium and integrin binding protein2). These genes encode proteins that are involved in hair bundle morphogenesis in the inner ear and are part of an interactome (see, for example, Mathur & Yang, 2015, Biochim. Biophys. Acta, 1852:406-20). Harmonin resides at the center of the USH1 interactome where it binds to other Usher 1 proteins. Because of its PDZ (PSD-59 95/Dlg/ZO-1) interaction domains, harmonin has been proposed to function as a scaffolding protein. In vitro binding studies have shown that all other known USH1 proteins bind to PDZ domains of harmonin as do two of the USH2 proteins, usherin, and VLGR1. The USH1 C gene consists of 28 exons, which code for 10 alternative splice forms of harmonin, grouped into three different subclasses (a, b and c) depending on the domain composition of the protein. The three isoforms differ in the number of PDZ protein-protein interaction domains, coiled-coiled (CC) domains, and proline-serine-threonine (PST) rich domains. USH1 proteins are localized to the apex of hair cells in mechanosenosory hair bundles, which are composed of hundreds of stereocilia interconnected by numerous extracellular links. Cadherin 23 and Protocadherin 15, products of Usher genes (USHI D and USH1 E, respectively) form tip-links located at the distal end of the stereocilia. Harmonin-b binds to CDH23, PCDH15, F-actin and itself. It is found at the tips of the stereocilia near the tip-link insertion point in hair cells where it is thought to play a functional role in transduction and adaptation in hair cells. Harmonin-b is expressed during early postnatal stages but its expression diminishes around postnatal day 30 (P30) in both the cochlea and vestibule. Harmonin-a also binds to cadherin 23 and is found in the stereocilia. Recent reports reveal an additional role for harmonin-a at the synapse where it associates with Cavl.3 Ca2+ channels to limit channel availability through a ubiquitin-dependent pathway.

Several mouse models for Usher syndrome have been identified or engineered over the past decade, seven of which affect harmonin. Of these, only one model, the Ush 1 c c.216G>A model, reproduces both auditory and retinal deficits that characterize human Usher Syndrome. Ushlc c.216G>A is a knock-in mouse model that affects expression of all conventional harmonin isoforms due a point mutation similar to the one found in a cohort of French-Acadian USHI C patients. The mutation introduces a cryptic splice site at the end of exon three of the Ushl c gene. Use of this cryptic splice site produces a frame-shifted transcript with a 35 bp deletion and results in translation of a severely truncated protein lacking PDZ, PST and CC domains. Homozygous C.216AA knock-in mice suffer from severe hearing loss at 1 month of age while heterozygous C.216GA mice do not present any abnormal phenotype. Cochlear histology in C.21 AA mice shows disorganized hair bundles, abnormal cell rows and loss of both inner and outer hair cells in middle and basal turns at P30.

Over 40 distinct mutations have been identified in TMC1 that cause deafness. These are subdivided into 35 recessive mutations and 5 dominant mutations. Most of the recessive mutations cause profound, congenital hearing loss (e.g., DFNB7/1 1) though a few cause later onset, moderate to severe hearing loss. All of the dominant mutations cause progressive hearing loss (e.g., DFNA36), with onset in the mid-teen years. In particular, an AAV vector that includes an Anc80 capsid protein as described herein can be used to deliver a non-mutant (e.g., wild type) TMC1 sequence or TMC2 sequence, thereby preventing hearing loss (e.g., further hearing loss) and/or restoring hearing function.

Therapeutic gene transfer to the cochlea has been considered to further improve upon the current standard of care ranging from age-related and environmentally induced hearing loss to genetic forms of deafness. More than 300 genetic loci have been linked to hereditary hearing loss with over 70 causative genes described (Parker & Bitner-Glindzicz, 2015, Arch. Dis. Childhood, 100:271-8). Therapeutic success in these approaches relies significantly on the safe and efficient delivery of exogenous gene constructs to the relevant therapeutic cell targets in the organ of Corti (OC) in the cochlea. The OC includes two classes of sensory hair cells: IHCs, which convert mechanical information carried by sound into electrical signals transmitted to neuronal structures and OHCs which serve to amplify and tune the cochlear response, a process required for complex hearing function. Other potential targets in the inner ear include spiral ganglion neurons, columnar cells of the spiral limbus, which are important for the maintenance of the adjacent tectorial membrane or supporting cells, which have protective functions and can be triggered to trans-differentiate into hair cells up to an early neonatal stage.

As used herein, inner ear cells refer to, without limitation, inner hair cells (IHCs), outer hair cells (OHCs), spiral ganglion neurons, stria vascularis, vestibular hair cells, vestibular ganglion neurons, and supporting cells. Supporting cells refer to cells in the ear that are not excitable, e.g., cells that are not hair cells or neurons. An example of a supporting cell is a Schwann cell.

Any suitable cell or mammal can be administered or treated by a method or use described herein. Typically, a mammal in need of a method described herein is suspected of having or expressing an abnormal or aberrant protein that is associated with a disease state. Alternative, the mammalian recipient may have a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects) and acquired pathologies (i.e., a pathological condition which is not attributable to an inborn defect). Accordingly, as used herein, the term “therapeutic agent” refers to any agent or material, which has a beneficial effect on the mammalian recipient. Thus, “therapeutic agent” embraces both therapeutic and prophylactic molecules having nucleic acid or protein components.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles to a mammal as set forth herein, where severity, frequency, progression or time of onset of one or more symptoms of a disease state, such as a neuro-degenerative disease, decreased, reduced, prevented, inhibited or delayed. In certain embodiments, a method includes administering a plurality of viral particles to a mammal to treat an adverse symptom of a disease state, such as a neuro-degenerative disease. In certain embodiments, a method includes administering a plurality of viral particles to a mammal to stabilize, delay or prevent worsening, or progression, or reverse and adverse symptom of a disease state, such as a neuro-degenerative disease.

In certain embodiments a method includes administering a plurality of viral particles to the central nervous system, or portion thereof as set forth herein, of a mammal and severity, frequency, progression or time of onset of one or more symptoms of a disease state, such as a neuro-degenerative disease, are decreased, reduced, prevented, inhibited or delayed by at least about 5 to about 10, about 10 to about 25, about 25 to about 50, or about 50 to about 100 days.

In some embodiments, a composition comprising a therapeutically effective number of virus particles containing a transgene, or containing one or more sets of different virus particles, wherein each particle in a set can contain the same type of transgene, but wherein each set of particles contains a different type of transgene than in the other sets, as described herein can be delivered.

Formulations according to the present invention can be used for CNS delivery via various techniques and routes including, but not limited to, intraparenchymal, intracerebral, intravetricular cerebral (ICV), intrathecal (e.g., IT-Lumbar, IT-thoracic, IT-cisterna magna) administrations and any other techniques and routes for injection directly or indirectly to the CNS and/or CSF.

In some embodiments, a formulation is delivered to the CNS by administering into the cerebrospinal fluid (CSF) of a subject in need of treatment. In some embodiments, intrathecal administration is used to deliver viral particles into the CSF. As used herein, intrathecal administration (also referred to as intrathecal injection) refers to an injection into the spinal canal (intrathecal space surrounding the spinal cord). Various techniques may be used including, without limitation, lateral cerebroventricular injection through a burrhole or cisternal or lumbar puncture or the like. Exemplary methods are described in Lazorthes et al. Advances in Drug Delivery Systems and Applications in Neurosurgery, 143-192 and Omaya et al., Cancer Drug Delivery, 1: 169-179, the contents of which are incorporated herein by reference.

According to the present invention, viral particles may be injected at any region surrounding the spinal canal. In some embodiments, viral particles are injected into the lumbar area or the cisterna magna or intraventricularly into a cerebral ventricle space. As used herein, the term “lumbar region” or “lumbar area” refers to the area between the third and fourth lumbar (lower back) vertebrae and, more inclusively, the L2-S 1 region of the spine. Typically, intrathecal injection via the lumbar region or lumber area is also referred to as “lumbar IT delivery” or “lumbar IT administration.” The term “cisterna magna” refers to the space around and below the cerebellum via the opening between the skull and the top of the spine. Typically, intrathecal injection via cisterna magna is also referred to as “cisterna magna delivery.” The term “cerebral ventricle” refers to the cavities in the brain that are continuous with the central canal of the spinal cord. As such, intrathecal administration includes any infusion into the central canal. Typically, injections via the cerebral ventricle cavities are referred to as intravetricular cerebral (ICV) delivery.

Various devices may be used for intrathecal delivery according to the present invention. In some embodiments, a device for intrathecal administration contains a fluid access port (e.g., injectable port); a hollow body (e.g., catheter) having a first flow orifice in fluid communication with the fluid access port and a second flow orifice configured for insertion into spinal cord; and a securing mechanism for securing the insertion of the hollow body in the spinal cord. In various embodiments, the fluid access port comprises a reservoir. In some embodiments, the fluid access port comprises a mechanical pump (e.g., an infusion pump). In some embodiments, an implanted catheter is connected to either a reservoir (e.g., for bolus delivery), or an infusion pump. The fluid access port may be implanted or external

In some embodiments, intrathecal administration may be performed by either lumbar puncture (i.e., slow bolus) or via a port-catheter delivery system (i.e., infusion or bolus). In some embodiments, the catheter is inserted between the laminae of the lumbar vertebrae and the tip is threaded up the thecal space to the desired level (generally L3-L4).

A single dose volume suitable for intrathecal administration is typically small. Typically, intrathecal delivery according to the present invention maintains the balance of the composition of the CSF as well as the intracranial pressure of the subject. In some embodiments, intrathecal delivery is performed absent the corresponding removal of CSF from a subject. In some embodiments, a suitable single dose volume may be e.g., less than about 10 ml, 8 ml, 6 ml, 5 ml, 4 ml, 3 ml, 2 ml, 1.5 ml, 1 ml, or 0.5 ml. In some embodiments, a suitable single dose volume may be about 0.5-5 ml, 0.5-4 ml, 0.5-3 ml, 0.5-2 ml, 0.5-1 ml, 1-3 ml, 1-5 ml, 1.5-3 ml, 1-4 ml, or 0.5-1.5 ml. In some embodiments, intrathecal delivery according to the present invention involves a step of removing a desired amount of CSF first. In some embodiments, less than about 10 ml (e.g., less than about 9 ml, 8 ml, 7 ml, 6 ml, 5 ml, 4 ml, 3 ml, 2 ml, 1 ml) of CSF is first removed before IT administration. In those cases, a suitable single dose volume may be e.g., more than about 3 ml, 4 ml, 5 ml, 6 ml, 7 ml, 8 ml, 9 ml, 10 ml, 15 ml, or 20 ml.

Various other devices may be used to effect intrathecal administration of a therapeutic composition. For example, formulations containing desired enzymes may be given using an Ommaya reservoir which is in common use for intrathecally administering drugs for meningeal carcinomatosis (Lancet 2: 983-84, 1963). More specifically, in this method, a ventricular tube is inserted through a hole formed in the anterior horn and is connected to an Ommaya reservoir installed under the scalp, and the reservoir is subcutaneously punctured to intrathecally deliver the particular enzyme being replaced, which is injected into the reservoir. Other devices for intrathecal administration of therapeutic compositions or formulations to an individual are described in U.S. Pat. No. 6,217,552, incorporated herein by reference. Alternatively, the viral particles may be intrathecally given, for example, by a single injection, or continuous infusion. It should be understood that the dosage treatment may be in the form 15 of a single dose administration or multiple doses.

In one embodiment of the invention, the viral particles are administered by lateral cerebro ventricular injection into the brain of a subject. The injection can be made, for example, through a burr hole made in the subject's skull. In another embodiment, the viral particles and/or other pharmaceutical formulation are administered through a surgically inserted shunt into the cerebral ventricle of a subject. For example, the injection can be made into the lateral ventricles, which are larger. In some embodiments, injection into the third and fourth smaller ventricles can also be made. In yet another embodiment, the pharmaceutical compositions used in the present invention are administered by injection into the cisterna magna, or lumbar area of a subject.

### IV. PHARMACEUTICAL COMPOSITIONS

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of viral particles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of viral particles can optionally include one or more of the following components: a sterile diluent such as water for injection, saline solution, such as phosphate buffered saline (PBS), artificial CSF, a surfactants, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, MD; and Poznansky et al., Drug Delivery Systems (1980), R. L. Juliano, ed., Oxford, N.Y., pp. 253-315).

Viral particles and their compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the number of viral particles believed necessary to produce the desired effect(s). The amount necessary can be formulated in a single dose, or can be formulated in multiple dosage units. The dose may be adjusted to a suitable viral particle concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms or an adverse effect of a disease state in question or an amount sufficient to confer the desired benefit.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, and pharmaceutical compositions thereof, can be packaged in single or multiple unit dosage form for ease of administration and uniformity of dosage.

Formulations containing viral particles typically contain an effective amount, the effective amount being readily determined by one skilled in the art. The viral particles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### V. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, IRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. A pol II promoter includes a minimal promoter that is a short DNA sequence comprised of a TATA-box and optionally other sequences that serve to specify the site of transcription initiation, to which regulatory elements are added for control of expression. A type 1 pol III promoter includes three cis-acting sequence elements downstream of the transcriptional start site: a) 5′sequence element (A block); b) an intermediate sequence element (I block); c) 3′ sequence element (C block). A type 2 pol III promoter includes two essential cis-acting sequence elements downstream of the transcription start site: a) an A box (5′ sequence element); and b) a B box (3′ sequence element). A type 3 pol III promoter includes several cis-acting promoter elements upstream of the transcription start site, such as a traditional TATA box, proximal sequence element (PSE), and a distal sequence element (DSE).

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples of promoters include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, actin promoter, U6, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

Transgenes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

In certain embodiments, cell-type-specific or inducible promoters, enhancers and the like, are employed in the methods and uses described herein. Non-limiting examples of cell-type-specific promoters include those isolated from the genes from TMC1, TMC2, Espin, PCDH15, PTPRQ, TMHS (LHFPL5), MYOIA. Non-limiting examples of inducible promoters include DNA responsive elements for ecdysone, tetracycline, hypoxia and IFN.

In certain embodiments, an expression control element comprises a CMV enhancer. In certain embodiments, an expression control element comprises a beta actin promoter. In certain embodiments, an expression control element comprises a chicken beta actin promoter. In certain embodiments, an expression control element comprises a CMV enhancer and a chicken beta actin promoter.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors, to the central nervous system, such as to distinct brain regions.

A recombinant virus so modified may preferentially bind to one type of tissue (e.g., CNS tissue) over another type of tissue (e.g., liver tissue). In certain embodiments, a recombinant virus bearing a modified capsid protein may “target” brain vascular epithelia tissue by binding at level higher than a comparable, unmodified capsid protein. For example, a recombinant virus having a modified capsid protein may bind to brain vascular epithelia tissue at a level 50% to 100% greater than an unmodified recombinant virus.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

By “mechanosensation” is meant a response to a mechanical stimulus. Touch, hearing, and balance of examples of the conversion of a mechanical stimulus into a neuronal signal. Mechanosensory input is converted into a response to a mechanical stimulus through a process termed “mechanotransduction.”

By “disease” is meant any condition or disorder that damages or interferes with the normal function of a cell, tissue, or organ. Examples of diseases include genetic disorders characterized by a loss of function in a protein that functions in mechanosensory transduction that is expressed, for example, in the inner ear of a subject. In another embodiment, the disease is Usher Syndrome (e.g., USH1) or age-related hearing loss. In one embodiment, a disease is an auditory disorder associated with a genetic defect, such as a defect in TMC1, TMC2, MY07A, USCHI C, CDH23, PCDH15, SANS, CIB2, USH2A, VLGR1, WHKN, CLRN1, PDZD7, USHIC (e.g., harmonin-a, b, or c).

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### VI. KITS

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, and/or viral particles.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD-or DVD-ROM/RAM, DVD, MP3, or an electrical storage media such as RAM and ROM or hybrids of these such as magnetic/optical storage media, FLASH memory, hybrids and memory type cards.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Delivery of AAV Vectors to the Cochlea

Gene therapy is a powerful tool to combat hearing loss and deafness, but its clinical implementation is impeded by the temporal bone anatomy, a high diversity of genetic lesions, and myriad cochlear sensory- and supporting-cell types. The identification of adeno-associated virus (AAV) capsids capable of efficient hair-cell transduction, including Anc80L65 and AAV2, has brought cochlear gene therapy closer to a clinical reality for some forms of genetic deafness. However, for genetic lesions impacting hearing at the level of cochlear supporting cells, additional vectors with varying tropism characteristics are needed. To meet this need, the inventors implemented a directed AAV evolution screen in the nonhuman primate (NHP) cochlea to identify a library of AAV capsid variants with diverse transduction properties capable of satisfying the requirements of current and future cochlear gene therapy designs.

Peptide modified AAV libraries were generated by insertion of random peptides into AAV1, AAV2, and AAV9 by insertion of random sequences at position 590 of AAV1 capsid, position 587 of AAV2 capsid, and position 588 of AAV9 capsid, respectively. A cochlea-specific AAV enrichment study was performed in which the AAV capsid library (3E11 vg) was delivered directly to the cochlea via the same RWM+LCF method. An eGFP expression construct was packaged into the AAV, driven by the CAG promoter. Four cochlea (two NHPs) received injection of AAV capsid library over the course of two rounds of in vivo enrichment.

At each round of enrichment NGS amplicon sequencing libraries were generated and sequencing data was used to track enrichment of each capsid configuration. Initially, the AAV capsid library was delivered to the NHP brain by intracerebroventricular (ICV) injection. In this experiment, striking and unexpected transduction of NHP cochlear hair-cells and supporting cells by AAV9 was observed. This unexpected transduction of cochlear cells following ICV injection is possible due to the fluid connection of the CSF to the perilymph via the cochlear aqueduct.

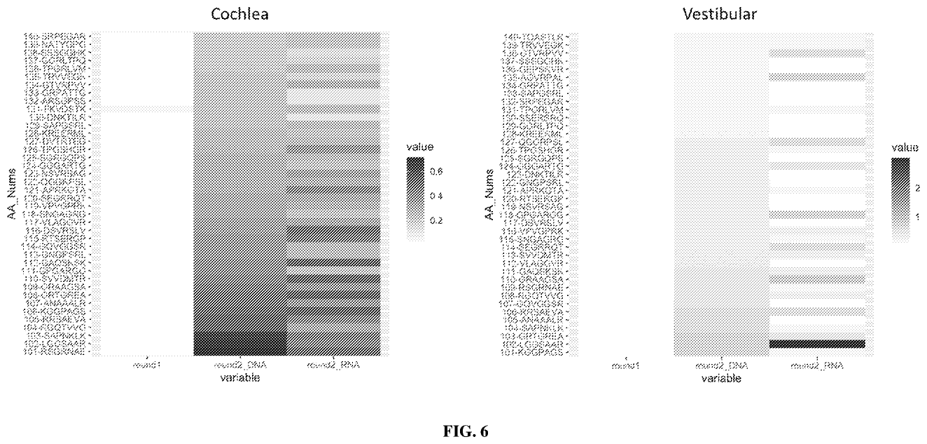

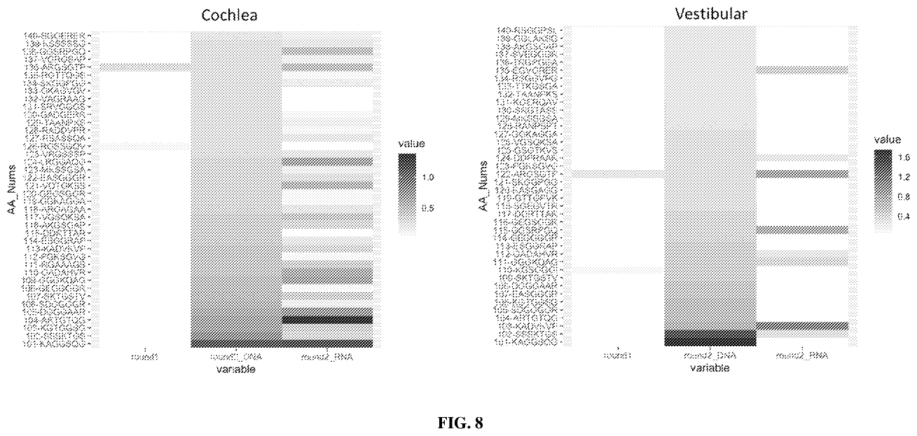

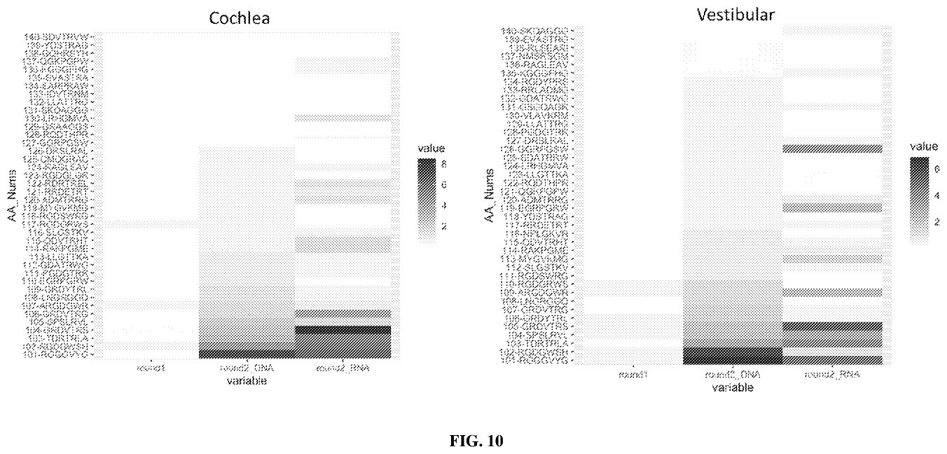

Round-over-Round enrichment heatmaps were generated. These illustrate the enrichment of indicated barcodes at baseline (round 0), and after rounds one and two of in vivo passaging through rhesus macaque. To generate these, the fastq results files for each tissue and round combination were processed using a custom Python script designed to extract and quantify unique barcode configurations observed at the DNA level. A custom R script was used to calculate the percentages of barcodes present in each sample and convert DNA barcodes to amino acid barcodes. FIG. 6 corresponds to samples treated with the AAV1-derived library; FIG. 8 corresponds to samples treated with the AAV2-derived library; FIG. 10 corresponds to samples treated with the AAV9-derived library.

Wild-type and modified AAV vectors were successfully delivered to the cochlea of Rhesus macaque. AAV vectors are able to transduce cochlear inner hair cells, as well as an assortment of supporting cells including cells of the stria-vascularis and spiral ganglion neurons. Sequencing results from cochlear AAV enrichment reveal an assortment of enriched capsid variants for which small-library- and fluorescence-based validation is ongoing.

### Example 2—Therapeutic Delivery to the Cochlea Via the CSF

There is a need for non-invasive and non-surgical methods that facilitate cochlear therapeutic delivery. Herein, the inventors report that robust cochlear delivery has been achieved via ICV delivery of AAV through the CSF in rhesus macaque and mouse. This CSF-based method of cochlear therapeutic delivery is facilitated by diffusion of therapeutic particles through the cochlear aqueduct, an unobstructed fluid connection of the CSF in the brain to the perilymph filled scala vestibuli and scala tympani of the cochlea. This finding opens the door to use of a CSF route of delivery for cochlear therapeutics for humans in a clinical setting.

In a clinical setting it may be advantageous to deliver AAVs or other therapeutics for cochlea transduction to the CSF via lumbar puncture. This is a non-surgical outpatient procedure that can be completed in minutes.

In a clinical setting it may be advantageous to deliver AAVs or other therapeutics for cochlea transduction to the CSF via intracerebroventricular (ICV) injection. For this procedure a hole will be made in the skull and an injection needle will be precisely inserted targeting the ventricular space. While more invasive than a lumbar puncture this procedure is less invasive than some middle ear surgical approaches.

In a clinical setting it may be advantageous to deliver AAVs or other therapeutics for cochlea transduction to the CSF via other methods which access the CSF space and facilitate diffusion of AAV capsids or other therapeutic agents into the cochlea.

In a clinical setting it may be advantageous to deliver AAVs or other therapeutics for cochlea transduction to the CSF via intra cisterna magna or general intrathecal, which access the CSF space and facilitate diffusion of AAV capsids or other therapeutic agents into the cochlea.

### Example 3—AAV Evolution after Cochlea Direct Injection

To assess the enrichment of AAV capsid candidates across successive rounds of in vivo capsid library evolution, the percentage contribution of UMIs from each capsid barcode was visualized as a heatmap (FIGS. 6, 8, and 10). In each heatmap, from left to right, the columns represented are DNA extracted from round1, DNA extracted from round 2, and RNA extracted from round 2. The percentage contribution of the indicated capsid candidates can be seen increasing from column 1 (round 1 DNA) to column 2 (round 2 DNA). Detection of high levels of capsid in the round 2 RNA samples (column 3) was more variable among the top ranked round 2 DNA capsid candidates. Both DNA and RNA performance are valuable indicators of capsid candidate transduction properties and so care was taken to select capsid candidates for validation that performed well in both round 2 DNA and RNA categories. Capsid candidate LGGSAAR (SEQ ID NO: 39) was selected from among the AAV1 derived capsid candidates for its strong DNA and RNA performance in both Cochlea and pooled vestibular tissues (FIG. 6). Capsid candidate KAGGSQG (SEQ ID NO: 84) was selected for its strong DNA and RNA performance in cochlea despite its reduced RNA performance in vestibular tissues (FIG. 8). No capsid candidates were selected from AAV9 as a limited number of fluorescence validation positions (8) are available and other criteria were also considered (FIG. 10).

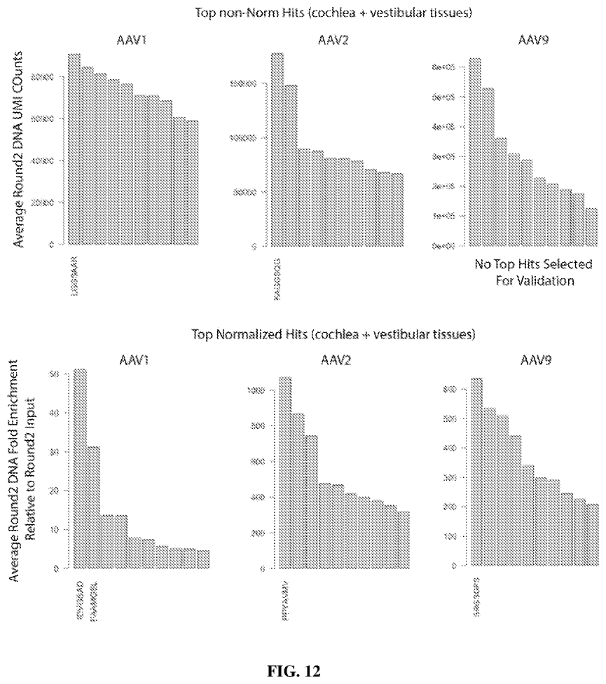

Additional capsid candidates were selected from rankings of the average UMI counts as well as the fold enrichment of % Round 2 DNA UMI counts over % DNA UMI counts obtained from the Round2 Input Vector Pool (FIG. 12).

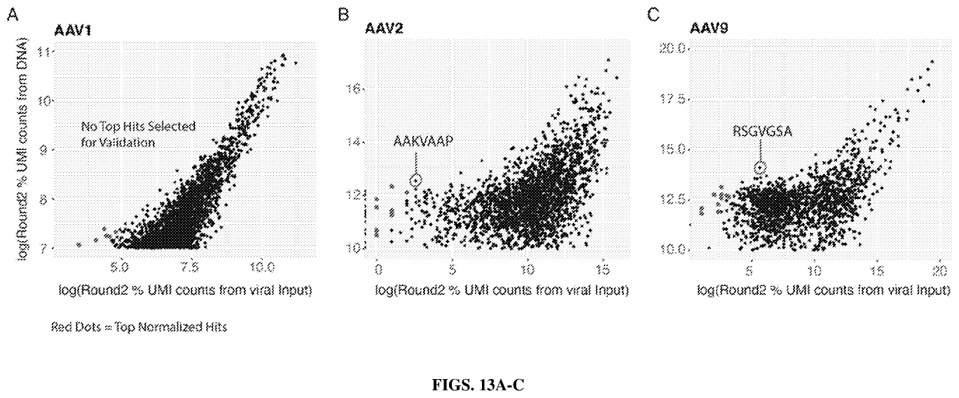

To select additional capsid candidates with favorable round 2 detection to round 2 input ratios, a scatter plot method was used to visualize the distribution of all capsid candidates. Generally, there is a tendency for candidates that are highly abundant in the round 2 input capsid library to be the most highly detected in the round 2 output library by raw UMI counts. There is also a tendency for the most highly enriched capsids after normalization to be those with very low detection in the Round2 input capsid library. Several candidates with low, but not the lowest, round2 input levels, which also had robust enrichment, were also selected. To this end, promising outlier candidates were identified from the scatterplot distributions. No capsid candidates were selected using this method from AAV1 (FIG. 13A), however AAV2 derived candidate AAKVAAP (SEQ ID NO: 154) (FIG. 13B), and AAV9 derived candidate RSGVGSA (SEQ ID NO: 155) (FIG. 13C) were selected using this method.

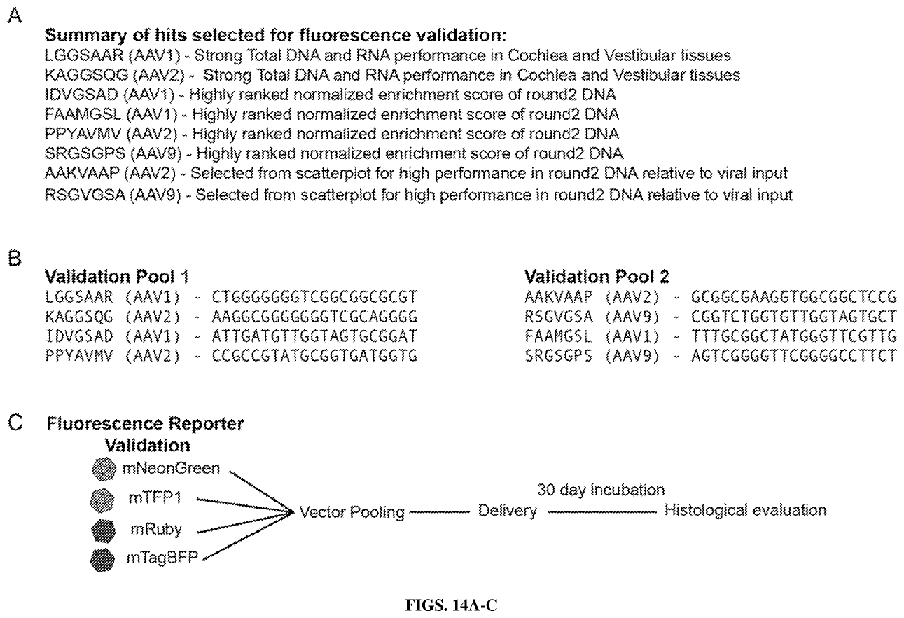

All capsid candidates selected to be carried forward into fluorescence validation are listed in FIG. 14A with their indicated parental serotype and a description of the criteria used to select that capsid. To facilitate delivery of separately fluorescently tagged capsid, one into each ear of a non-human primate, capsids were grouped into two validation pools (FIG. 14B). Capsid candidates were individually generated to deliver a fluorescence reporter expression construct. Capsids were pooled in groups of 4 to create validation pools. One validation pool will be delivered by direct intra-cochlear injection to each inner ear of a Rhesus Macaque. Thirty days post injection the animal will be sacrificed and cochlea collected for histological evaluation (FIG. 14C).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Kawamoto et al., The functional and structural outcome of inner ear gene transfer via the vestibular and cochlear fluids in mice. Mol Ther, 2001. 4(6): p. 575-85.
2. Yoshimura et al., Enhanced viral-mediated cochlear gene delivery in adult mice by combining canal fenestration with round window membrane inoculation. Sci Rep, 2018. 8(1): p. 2980.
3. Omichi et al., Hair Cell Transduction Efficiency of Single- and Dual-AAV Serotypes in Adult Murine Cochleae. Mol Ther Methods Clin Dev, 2020. 17: p. 1167-1177.
4. Suzuki et al., Cochlear gene therapy with ancestral AAV in adult mice: complete transduction of inner hair cells without cochlear dysfunction. Sci Rep, 2017. 7: p. 45524.
5. Stover et al., Transduction of the contralateral ear after adenovirus-mediated cochlear gene transfer. Gene Ther, 2000. 7(5): p. 377-83.
6. Rosenberg et al., Single-cell profiling of the developing mouse brain and spinal cord with split-pool barcoding. Science 360:176-182, 2018.

What is claimed is:

1. A method to deliver a therapeutic transgene to the cochlea of a subject, comprising administering to the cerebrospinal fluid of the subject a modified adeno-associated virus (AAV) encoding a therapeutic transgene that treats or prevents a hearing or vestibular disorder when expressed in a cell of the cochlea.

2. The method of claim 1, wherein the therapeutic transgene is operably linked to a cochlea-specific promoter.

3. The method of claim 2, wherein the cochlea-specific promoter is a hair cell-specific promoter.

4. The method of claim 2, wherein the cochlea-specific promoter is a support cell-specific promoter, such as a GJB2 promoter.

5. The method of any one of claims 1-4, wherein the modified AAV comprises a modified capsid protein.

6. The method of any one of claims 1-5, wherein the modified capsid protein comprises a targeting peptide, wherein the targeting peptide is three to ten amino acids in length.

7. The method of any one of claims 1-6, wherein the modified AAV capsid protein is a modified AAV1 capsid protein, a modified AAV2 capsid protein, or a modified AAV9 capsid protein.

8. The method of claim 7, wherein the modified AAV capsid protein is derived from an AAV1 capsid protein, wherein the targeting peptide is inserted after residue 590 of the AAV1 capsid protein.

9. The method of claim 8, wherein the targeting peptide is selected from SEQ ID NOs: 150, 151, and 1-44.

10. The method of claim 8, wherein the targeting peptide is selected from SEQ ID NOs: 39, 150, and 151.

11. The method of any one of claims 8-10, wherein the targeting peptide is flanked by linker sequences, wherein the linker sequences on each side of the targeting peptides are two or three amino acids long.

12. The method of claim 11, wherein the linker sequences are SSA on the N-terminal side of the targeting peptide and AS on the C-terminal side of the targeting peptide.

13. The method of claim 7, wherein the modified AAV capsid protein is derived from an AAV2 capsid protein, wherein the targeting peptide is inserted after residue 587 of the AAV2 capsid protein.

14. The method of claim 13, wherein the targeting peptide is selected from SEQ ID NOs: 152, 154, and 45-100.

15. The method of claim 13, wherein the targeting peptide is selected from SEQ ID NOs: 84, 152, and 154.

16. The method of any one of claims 13-15, wherein the targeting peptide is flanked by linker sequences, wherein the linker sequences on each side of the targeting peptides are two or three amino acids long.

17. The method of claim 16, wherein the linker sequences are AAA on the N-terminal side of the targeting peptide and AA on the C-terminal side of the targeting peptide.

18. The method of claim 7, wherein the modified AAV capsid protein is derived from an AAV9 capsid protein, wherein the targeting peptide is inserted after residue 588 of the AAV9 capsid protein.

19. The method of claim 18, wherein the targeting peptide is selected from SEQ ID NOs: 153, 155, and 101-149.

20. The method of claim 18, wherein the targeting peptide is selected from SEQ ID NOs: 153 and 155.

21. The method of any one of claims 18-20, wherein the targeting peptide is flanked by linker sequences, wherein the linker sequences on each side of the targeting peptides are two or three amino acids long.

22. The method of claim 21, wherein the linker sequences are AAA on the N-terminal side of the targeting peptide and AS on the C-terminal side of the targeting peptide.

23. The method of any one of claims 6-22, wherein the targeting peptide is seven amino acids in length.

24. The method of any one of claims 1-23, wherein the therapeutic transgene is an siRNA, shRNA, miRNA, non-coding RNA, lncRNA, therapeutic protein, or CRISPR system.

25. The method of any one of claims 1-24, wherein the administration is to a cisterna magna, an intraventricular space, a brain ventricle, a subarachnoid space, and/or an intrathecal space.

26. The method of any one of claims 1-25, wherein the method delivers the therapeutic transgene to a cell of the inner ear.

27. The method of claim 26, wherein the cell in the inner ear is selected from the group consisting of spiral ganglion neurons, vestibular hair cells, vestibular ganglion neurons, supporting cells, and cells in the stria vascularis.

28. The method of claim 26, wherein the cell is a hair cell of the cochlea or vestibular system.

29. The method of claim 28, wherein the cell is an inner hair cell of the cochlea or an outer hair cell of the cochlea.

30. The method of claim 29, wherein the subject has a hearing disorder, and the molecular therapeutic is delivered in a therapeutically effective amount.

31. The method of claim 29, wherein the subject is at risk of exposure to damaging auditory stimuli.

32. The method of claim 30 or 31, wherein the therapeutic transgene is delivered to at least 80% of inner hair cells and/or at least 80% of outer hair cells.

33. The method of claim 30 or 31, wherein the administering reverses or prevents hearing loss.

34. The method of claim 33, wherein the hearing loss is partial hearing loss or complete deafness.

35. The method of claim 28, wherein the cell of the vestibular system is a hair cell of the utricle, or a cell in an ampulla of a lateral semicircular canal, or a hair cell in a cupula.

36. The method of claim 28, wherein the cell is a cell of the vestibular system, the subject has a disorder of the vestibular system, and the transgene is delivered in a therapeutically effective amount.

37. The method of any one of claims 1-32, wherein the method treats or prevents hearing loss in a subject.

38. The method of any one of claims 1-32, wherein the method treats hereditary hearing loss in the subject.

39. The method of any one of claims 1-32, wherein the method treats or prevents impaired balance or impaired vestibular function in the subject.

40. The method of any one of claims 1-39, wherein a plurality of viral particles are administered.

41. The method of claim 40, wherein the virus is administered at a dose of about 1×10⁶ to about 1×10¹⁸ vector genomes per kilogram (vg/kg).

42. The method of claim 40, wherein the virus is administered at a dose from about 1×10⁷-1×1017, about 1×108-1×1016, about 1×109-1×1015, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², about 1×10¹²-×10¹³, or about 1×10¹³-1×10¹⁴ vg/kg of the patient.

43. The method of any one of claims 1-42, wherein the subject is human.

44. A modified adeno-associated virus (AAV) comprising a therapeutic transgene operably linked to a cochlea-specific promoter.

45. The modified AAV of claim 44, wherein the therapeutic transgene treats or prevents a hearing or vestibular disorder.

46. The modified AAV of claim 44, wherein the cochlea-specific promoter is a hair cell-specific promoter.

47. The modified AAV of claim 44, wherein the cochlea-specific promoter is a support cell-specific promoter, such as a GJB2 promoter.

48. The modified AAV of any one of claims 44-47, wherein the modified AAV comprises a modified capsid protein.

49. The modified AAV of claim 48, wherein the modified capsid protein comprises a targeting peptide, wherein the targeting peptide is three to ten amino acids in length.

50. The modified AAV of claim 48 or 49, wherein the modified AAV capsid protein is a modified AAV1 capsid protein, a modified AAV2 capsid protein, or a modified AAV9 capsid protein.

51. The modified AAV of claim 50, wherein the modified AAV capsid protein is derived from an AAV1 capsid protein, wherein the targeting peptide is inserted after residue 590 of the AAV1 capsid protein.

52. The modified AAV of claim 51, wherein the targeting peptide is selected from SEQ ID NOs: 150, 151, and 1-44.

53. The modified AAV of claim 51, wherein the targeting peptide is selected from SEQ ID NOs: 39, 150, and 151.

54. The modified AAV of any one of claims 51-53, wherein the targeting peptide is flanked by linker sequences, wherein the linker sequences on each side of the targeting peptides are two or three amino acids long.

55. The modified AAV of claim 54, wherein the linker sequences are SSA on the N-terminal side of the targeting peptide and AS on the C-terminal side of the targeting peptide.

56. The modified AAV of claim 50, wherein the modified AAV capsid protein is derived from an AAV2 capsid protein, wherein the targeting peptide is inserted after residue 587 of the AAV2 capsid protein.

57. The modified AAV of claim 56, wherein the targeting peptide is selected from SEQ ID NOs: 152, 154, and 45-100.

58. The modified AAV of claim 56, wherein the targeting peptide is selected from SEQ ID NOs: 84, 152, and 154.

59. The modified AAV of any one of claims 56-58, wherein the targeting peptide is flanked by linker sequences, wherein the linker sequences on each side of the targeting peptides are two or three amino acids long.

60. The modified AAV of claim 59, wherein the linker sequences are AAA on the N-terminal side of the targeting peptide and AA on the C-terminal side of the targeting peptide.

61. The modified AAV of claim 50, wherein the modified AAV capsid protein is derived from an AAV9 capsid protein, wherein the targeting peptide is inserted after residue 588 of the AAV9 capsid protein.

62. The modified AAV of claim 61, wherein the targeting peptide is selected from SEQ ID NOs: 153, 155, and 101-149.

63. The modified AAV of claim 61, wherein the targeting peptide is selected from SEQ ID NOs: 153 and 155.

64. The modified AAV of any one of claims 61-63, wherein the targeting peptide is flanked by linker sequences, wherein the linker sequences on each side of the targeting peptides are two or three amino acids long.

65. The modified AAV of claim 64, wherein the linker sequences are AAA on the N-terminal side of the targeting peptide and AS on the C-terminal side of the targeting peptide.

66. The modified AAV of any one of claims 49-65, wherein the targeting peptide is seven amino acids in length.

67. A pharmaceutical composition comprising the modified AAV of any one of claims 44-66 and a pharmaceutically acceptable carrier.
